(19) United States
(12) Patent Application Publication
(10) Pub. No.: US 2004/0009949 A1
(43) Pub. Date: Jan. 15, 2004

(54) Method for treating autoimmune or inflammatory diseases with combinations of inhibitory oligonucleotides and small molecule antagonists of immunostimulatory CpG nucleic acids

(75) Inventor: Arthur M. Krieg, Wellesley, MA (US)

Correspondence Address: Alan W. Steele, M.D., Ph.D., Wolf, Greenfield & Sacks, P.C., 600 Atlantic Avenue, Boston, MA, 02210, US

(73) Assignee: Coley Pharmaceutical Group, Inc., Wellesley, MA (US)

(21) Appl. No.: 10/455,247

(22) Filed: Jun. 5, 2003

Related U.S. Application Data
(60) Provisional application No. 60/386,274, filed on Jun. 5, 2002.

(51) Int. Cl. A61K 48/00

(52) U.S. Cl. 514/44

(57) ABSTRACT

Improved methods are provided for inhibiting nucleic acid-induced immune activation and for treating autoimmune disease. The methods involve using an inhibitory nucleic acid in synergistic combination with a small molecule antagonist of immunostimulatory CpG nucleic acids. Inhibitory nucleic acids useful according to the invention include poly G nucleic acids. Small molecule antagonists of immunostimulatory CpG nucleic acids useful according to the invention include chloroquine and derivatives of chloroquine-like molecules, including substituted 2-phenylquinolin-4-amines.

### RELATED APPLICATIONS

This application claims benefit under 35 U.S.C. 119(e) of U.S. provisional patent application Serial No. 60/386,274, filed Jun. 5, 2002, the entire contents of which are incorporated herein by reference.

### FIELD OF THE INVENTION

The present invention relates to the fields of immunomodulatory nucleic acids and autoimmunity. More specifically, the present invention relates to inhibitory nucleic acid molecules, small molecule antagonists of immunostimulatory CpG nucleic acids, such as quinolines, and to diseases associated with molecular complexes, particularly immune complexes, containing nucleic acid. Such autoimmune diseases include, among others, rheumatoid arthritis (RA) and systemic lupus erythematosus (SLE).

### BACKGROUND OF THE INVENTION

Bacterial DNA is known to be mitogenic for mammalian B lymphocytes (B cells), while mammalian DNA is not. The mitogenicity of bacterial DNA has been attributed to the presence of CpG DNA, DNA containing unmethylated cytosine-guanine (CG) dinucleotides within a flanking base context referred to as a CpG motif. Synthetic oligodeoxynucleotides (ODN) containing CpG motifs have been shown to exert similar immunostimulatory effects as bacterial DNA. The immunostimulatory effects of CpG DNA include induction of B cell proliferation, immunoglobulin secretion, secretion of certain cytokines including IL-6, IL-12, interferon gamma (IFN-γ), secretion of certain chemokines including IFN-γ-inducible protein 10 (IP-10), and protection of B cells against apoptosis.

The mechanism of action underlying the immunostimulatory effects of CpG DNA was recently reported to involve signaling involving Toll-like receptor 9 (TLR9). TLR9, like other TLR family members, is believed to be a receptor associated with innate immunity, i.e., immunity that is responsive to certain molecular patterns characteristic of foreign pathogens. Through an unknown mechanism DNA is taken up by cells and directed into endosomes. It is believed that TLR9 is present in the endosomes and that an acidification or other endosomal maturation step is involved in CpG-DNA-induced TLR9 signaling.

Chloroquine, hydroxychloroquine, and quinacrine induce remission of SLE and RA by an unknown mechanism. These drugs are reported to bind to double-stranded DNA by intercalation. They are weak bases and they partition into acidic vesicles, i.e., endosomes. At high concentration, chloroquine can collapse the intravesicular pH gradient.

Recently others have reported that certain small molecules related to chloroquine, hydroxychloroquine, and quinacrine act as antagonists to immunostimulatory CpG oligodeoxynucleotides. U.S. Pat. Nos. 6,221,882; 6,399,630; 6,479,504; and 6,521,637; published PCT application PCT/US00/16723 (WO 00/76982); Strekowski L et al. (1999) Bioorg Med Chem Lett 9:1819-24, Strekowski L et al. (2003) J Med Chem 46:1242-9, and Strekowski L et al. (2003) Bioorg Med Chem 11:1079-85.

Recent studies have reported that certain ODN containing poly G sequences can inhibit the stimulatory effects of CpG DNA on B cells and macrophages. Lenert P et al. (2001) Antisense Nucleic Acid Drug Dev 11:247-56. It has also been reported that these inhibitory ODN can block the activation of rheumatoid factor-specific B cells by immune complexes that contain DNA. Leadbetter E A et al. (2002) Nature 416:603-7.

### SUMMARY OF THE INVENTION

The present invention is based in part on the surprising finding that a combination of an inhibitory nucleic acid and a chloroquine-like molecule, a 4-aminoquinoline, a 2-phenylquinoline, or other small molecule antagonist of immunostimulatory CpG nucleic acids has a synergistic activity to block disease-inducing effects of host or foreign DNA in vivo, and may therefore be useful in the treatment of autoimmune diseases. It was surprisingly discovered that, as opposed to their inhibitory effect on immune stimulatory nucleic acids, chloroquine and chloroquine-like molecules and other small molecule antagonists of immunostimulatory CpG nucleic acids do not interfere with the immune inhibitory effect of inhibitory nucleic acids. Rather, the inhibitory nucleic acid molecules and small molecule antagonists of immunostimulatory CpG nucleic acids act synergistically to block disease-inducing effects of either bacterial or other foreign nucleic acid, or immune complexes containing host nucleic acid that by itself normally would not be expected to trigger immune activation through TLR9. It is possible that one or both of the inhibitory nucleic acid and the small molecule antagonist of immunostimulatory CpG nucleic acids may directly bind to TLR9 and/or prevent the foreign nucleic acid or host nucleic acid/immune complex from binding to TLR9, or the inhibitory effect could also come at a downstream point in the TLR9 signaling pathway.

It is the belief of the applicant that methods of the instant invention are useful for the treatment and prevention of autoimmune and inflammatory diseases. In particular, the methods of the instant invention are useful for the treatment and prevention of autoimmune and inflammatory diseases in which a nucleic acid, or a complex containing a polypeptide and a nucleic acid, is recognized by the immune system as a danger signal. The complex containing a polypeptide and a nucleic acid can be an immune complex involving an antibody and a nucleic acid, or it can be a complex involving polypeptide, other than an antibody, and a nucleic acid.

It is the belief of the applicant that methods of the instant invention are particularly useful for the treatment of RA and SLE.

In one aspect of the invention, a method is provided for inhibiting immune activation. The method according to this aspect of the invention involves contacting a TLR9-expressing cell with an inhibitory nucleic acid and a small molecule antagonist of immunostimulatory CpG nucleic acids, in an effective amount to inhibit activation of the TLR9-expressing cell by a nucleic acid-containing molecular complex. The nucleic acid-containing molecular complex includes a nucleic acid molecule and a molecule other than an antibody. The molecule other than an antibody can be a protein or polypeptide, a lipid, a carbohydrate, or any combination thereof. The effective amount of the inhibitory nucleic acid and the small molecule antagonist of immunostimulatory CpG nucleic acids includes a synergistic amount of either one with respect to the other.

In one aspect of the invention, a method is provided for inhibiting immune activation. The method according to this aspect of the invention involves contacting a TLR9-expressing cell with an inhibitory nucleic acid and a small molecule antagonist of immunostimulatory CpG nucleic acids, in an effective amount to inhibit activation of the TLR9-expressing cell by a nucleic acid-containing immune complex. The nucleic acid-containing immune complex includes a nucleic acid molecule and an antibody. The effective amount of the inhibitory nucleic acid and the small molecule antagonist of immunostimulatory CpG nucleic acids includes a synergistic amount of either one with respect to the other.

The TLR9-expressing cell can be a cell that expresses TLR9 either naturally or artificially. In certain embodiments according to these first two aspects of the invention, the TLR9-expressing cell is chosen from a B cell, a dendritic cell, an endothelial cell, and a macrophage. In one embodiment the dendritic cell is a plasmacytoid dendritic cell (pDC). In a particular embodiment the TLR9-expressing cell is a B cell. In one embodiment the TLR9-expressing cell is a human cell.

In another aspect of the invention, a method is provided for treating an autoimmune disease. The method according to this aspect involves administering to a subject having or at risk of developing an autoimmune disease an inhibitory nucleic acid and a small molecule antagonist of immunostimulatory CpG nucleic acids, in an effective amount to treat or prevent the autoimmune disease. The effective amount of the inhibitory nucleic acid and the small molecule antagonist of immunostimulatory CpG nucleic acids includes a synergistic amount of either one with respect to the other.

In one embodiment according to this aspect of the invention, the autoimmune disease is chosen from rheumatoid arthritis (RA), systemic lupus erythematosus (SLE), inflammatory bowel disease (IBD), multiple sclerosis (MS), glomerulonephritis, type 1 diabetes mellitus, Sjögren's syndrome, viral infections associated with hepatitis B virus (HBV) and hepatitis C virus (HCV), graft-versus-host disease (GvHD), paraneoplastic autoimmune syndrome associated with small cell lung cancer, and paraneoplastic autoimmune syndrome associated with breast cancer.

The following apply to all aspects of the invention.

In some embodiments the inhibitory nucleic acid includes a poly G motif. In one embodiment the poly G motif includes a sequence chosen from GGGG; N₁GGGN₂GGGN₃ (SEQ ID NO:20), wherein N₁, N₂, and N₃ are each independently any nucleic acid sequence including 0-20 nucleotides; a sequence of 5 nucleotides in which at least 4 nucleotides are G; a sequence of 7 nucleotides in which at least 5 nucleotides are G; and a sequence of 8 nucleotides in which at least 6 nucleotides are G.

In some embodiments the inhibitory nucleic acid includes a sequence chosen from

| | |
|---|---|
| GTGCCGGGGTCTCCGGGC, | (SEQ ID NO:1) |
| GCTGTGGGGCGGCTCCTG, | (SEQ ID NO:2) |
| GGGGTCAACGTTGAGGGGGG, | (SEQ ID NO:3) |
| GGGGAGGGT, | (SEQ ID NO:4) |
| GGGGAGGGG, | (SEQ ID NO:5) |
| CACGTTGAGGGGCAT, | (SEQ ID NO:6) |
| TCCTGGCGGGGAAGT, | (SEQ ID NO:7) |
| TCCTGGAGGGGAAGT, | (SEQ ID NO:8) |
| GGCTCCGGGGAGGGAATTTTTGTCTAT, | (SEQ ID NO:9) |
| TCCTGCCGGGGAAGT, | (SEQ ID NO:10) |
| TCCTGCAGGGGAAGT, | (SEQ ID NO:11) |
| TCCTGAAGGGGAAGT, | (SEQ ID NO:12) |
| TCCTGGCGGGCAAGT, | (SEQ ID NO:13) |
| TCCTGGCGGGTAAGT, | (SEQ ID NO:14) |
| TCCTGGCGGGAAAGT, | (SEQ ID NO:15) |
| TCCGGGCGGGGAAGT, | (SEQ ID NO:16) |
| TCGGGGCGGGGAAGT, | (SEQ ID NO:17) |
| TCCCGGCGGGGAAGT, and | (SEQ ID NO:18) |
| GGGGGACGTTGGGGG. | (SEQ ID NO:19) |

In one embodiment the inhibitory nucleic acid has a stabilized backbone. In one embodiment the stabilized backbone is a phosphorothioate backbone.

In one embodiment the small molecule antagonist of immunostimulatory CpG nucleic acids is chosen from quinacrine, chloroquine, hydroxychloroquine, substituted 4-quinolinamines, 2-phenylquinolin-4-amines, 4-aminoquinolines, bis-4-aminoquinolines, and 9-aminoacridines (disclosed in U.S. Pat. Nos. 6,221,882; 6,399,630; 6,479,504; and 6,521,637).

In one embodiment the small molecule antagonist of immunostimulatory CpG nucleic acids is chosen from compounds having the structural Formula 1:

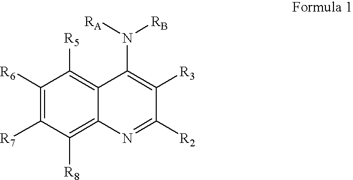

wherein R_{A} is a hydrogen atom, a lower alkyl group, or linked to R_{B} by a substituted or unsubstituted alkyl chain; R_{B} is a hydrogen atom, an alicyclic group, an alkyl secondary, tertiary or quaternary amine, or an alkenyl secondary, tertiary or quaternary amine; R₂ is a hydrogen atom, a lower alkyl group, an aryl group, a heteroaromatic group, or a lower alkenyl group substituted with an aryl group; R₃ is a hydrogen atom, a lower alkyl group, or an aromatic group; R₅ is a hydrogen atom, a lower alkyl group, or a halogen atom; R₆ is a hydrogen atom, a lower alkyl group, a lower alkoxy group, an aryloxy group, an aryl group, an amino group, or a thioether group; R₇ is a hydrogen atom, a lower alkyl group, a lower alkoxy group, an aryloxy group, a haloalkyl group, or a halogen atom; and R₈ is a hydrogen group, or a lower alkoxy group, and pharmaceutically acceptable salts thereof, with the proviso that if R₇ is a halogen, then at least one of R₂, R₃, R₅, R₆ or R₈ is non-hydrogen and R_{B} is not 4-[N,N-dialkyl-n-pentylamine] or 4-[N-alkyl-N-hydroxyalkyl-n-pentylamine].

In one embodiment the small molecule antagonist of immunostimulatory CpG nucleic acids is N-[3-(dimethylamino)propyl]-2-[4-(N-methylpiperazino)phenyl]quinolin-4-amine.

In one embodiment the small molecule antagonist of immunostimulatory CpG nucleic acids is N-[2-(dimethylamino)ethyl]-2-[4-(N-methylpiperazino)phenyl]quinolin-4-amine

In one embodiment the small molecule antagonist of immunostimulatory CpG nucleic acids is N-[4-(dimethylamino)butyl]-2-[4-(N-methylpiperazino)phenyl]quinolin-4-amine

In one embodiment the small molecule antagonist of immunostimulatory CpG nucleic acids is N,N′-Bis[4-[4-[2-(dimethylamino)ethyl]amino]quinolin-2-yl]phenyl]hexane-1,6-diamine

In one embodiment the small molecule antagonist of immunostimulatory CpG nucleic acids is N,N′-Bis[4-[4-[2-(dimethylamino)ethyl]amino]quinolin-2-yl]phenyl]-4,9-dioxa-1,12-dodecanediamine

In one embodiment the small molecule antagonist of immunostimulatory CpG nucleic acids is chosen from compounds having the structural Formula 2:

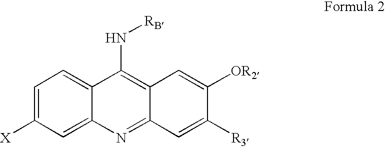

wherein R_{B′} is a hydrogen atom or an alkyl secondary, tertiary, or quaternary amino group; R_{2′} is a lower alkyl group; R_{3′} is a hydrogen atom or a lower alkoxy group; X is a halogen atom; and pharmaceutically acceptable salts thereof.

In some embodiments the small molecule antagonist of immunostimulatory CpG nucleic acids is a compound of Formula 3:

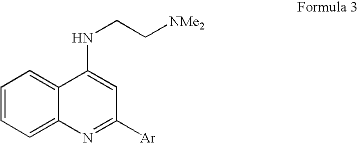

wherein Ar is selected from 2-naphthyl, 3-phenanthryl, 4-MePh, and trans-CH═CHPh. In one embodiment Ar is 2-naphthyl.

In some embodiments the small molecule antagonist of immunostimulatory CpG nucleic acids is a compound of Formula 4:

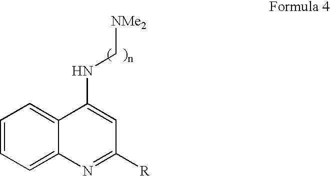

wherein n is an integer between 3 and 6, inclusive, and R is selected from p-tolyl or 2-naphthyl when n is 3, 2-naphthyl when n is 4, and 2-naphthyl when n is 6.

In some embodiments the small molecule antagonist of immunostimulatory CpG nucleic acids is a compound of Formula 5:

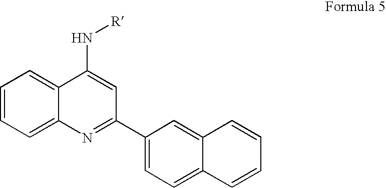

wherein R′ is (CH₂)₃N(CH₂CH₂)₂N(CH₂)₃NHC(O)(CH₂)₃OH.

In some embodiments the small molecule antagonist of immunostimulatory CpG nucleic acids is a compound of Formula 6:

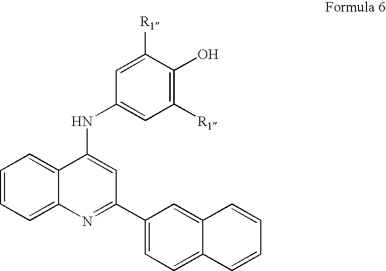

wherein R_{1″} is selected from morpholinomethyl, piperidinomethyl, pyrrolidinomethyl, and N-methylpiperazinomethyl. In one embodiment the small molecule antagonist of immunostimulatory CpG nucleic acids is a compound of Formula 6, wherein R_{1″} is N-methylpiperazinomethyl.

In one embodiment the small molecule antagonist of immunostimulatory CpG nucleic acids is chosen from bafilomycin A, monensin, concanamycin B, and ammonium chloride.

In one embodiment the nucleic acid-containing immune complex includes a CpG nucleic acid.

In some embodiments the nucleic acid-containing immune complex includes a nucleic acid from a foreign pathogen, i.e., from a source other than the host. The foreign pathogen can be a bacterium, a virus (including retrovirus), a fungus, or a parasite. In one embodiment the nucleic acid-containing immune complex includes a bacterial nucleic acid.

In one embodiment the nucleic acid-containing immune complex includes a host nucleic acid.

In one embodiment the nucleic acid-containing immune complex includes DNA. In various embodiments the nucleic acid-containing immune complex includes host DNA, host RNA, DNA binding protein, RNA binding protein (e.g., La, Ro, Sm), histone, chromatin, ribosomal protein, spliceosomal protein, and any combination thereof.

### BRIEF DESCRIPTION OF THE DRAWINGS

FIG. 1 is two paired bar graphs showing (A) the induction of NF-κB and (B) the amount of IL-8 produced by 293 fibroblast cells transfected with human TLR9 in response to exposure to various stimuli, including CpG-ODN, GpC-ODN, lipopolysaccharide (LPS), and medium.

FIG. 2 is a bar graph showing the induction of NF-κB produced by 293 fibroblast cells transfected with murine TLR9 in response to exposure to various stimuli, including CpG-ODN, methylated CpG-ODN (Me-CpG-ODN), GpC-ODN, LPS, and medium.

FIG. 3 is a series of gel images depicting the results of reverse transcriptase-polymerase chain reaction (RT-PCR) assays for murine TLR9 (mTLR9), human TLR9 (hTLR9), and glyceraldehyde-3-phosphate dehydrogenase (GAPDH) in untransfected control 293 cells, 293 cells transfected with mTLR9 (293-mTLR9), and 293 cells transfected with hTLR9 (293-hTLR9).

FIG. 4 is a graph showing the degree of induction of NF-κB-luc by various stimuli in stably transfected 293-hTLR9 cells.

FIG. 5 is a graph showing the degree of induction of NF-κB-luc by various stimuli in stably transfected 293-mTLR9 cells.

FIG. 6 is a graph depicting the concentration-dependent stimulation index TLR9-expressing cells by CpG ODN 2006 in the presence of buffer alone (solid circles), 0.05 μM inhibitory ODN 2088 alone (open circles), 0.078 μg/mL chloroquine alone (solid triangles), and ODN 2088 and chloroquine together (open triangles).

### DETAILED DESCRIPTION OF THE INVENTION

Chloroquine and related compounds hydroxychloroquine and quinacrine have been used in the treatment of RA and SLE for decades, but the mechanism of action has not really been understood, beyond the hypothesis that their effect on endosomal acidification may interfere with the presentation of autoantigens on major histocompatibility complex (MHC) molecules. Fox RI (1993) Semin Arthritis Rheum 23(2 Suppl 1):82-91. It has more recently been reported that chloroquine and certain related compounds, as well as unrelated compounds that also interfere with endosomal acidification or maturation, have strong inhibitory effects on the activation of the TLR9 pathway by immune stimulatory CpG DNA. Yi A K et al. (1998) J Immunol 160:4755-61; Hacker H et al. (1998) EMBO J 17:6230-40; Macfarlane D E et al. (1998) J Immunol 160:1122-31; Strekowski L et al. (1999) Bioorg Med Chem Lett 9:1819-24; Strekowski L et al. (2003) J Med Chem 46:1242-9; Strekowski L et al. (2003) Bioorg Med Chem 11:1079-85. The mechanism by which these compounds block the TLR pathway remains unclear. The different compounds described in these papers may all be working via the same mechanism, or through different mechanisms. DNA does not appear to interact with TLR9 at the cell surface, but instead is taken up into an endosomal subcompartment, which then appears to involve some sort of acidification or maturation step before the DNA can interact with TLR9. Some of these molecules may be inhibitors of a required endosomal step for the DNA to leave the endosome to interact with TLR9, or they may interfere with an interaction with TLR9 in the endosome, directly or indirectly.

It was recently reported that chloroquine and other compounds that block the effects of CpG DNA also block the activation of rheumatoid factor-specific B cells by immune complexes that contain DNA. Leadbetter E A et al. (2002) Nature 416:603-7. It is the belief of the applicant that chloroquine and related compounds such as those disclosed by Strekowski et al. are useful in the treatment of autoimmune diseases, especially those in which there are immune complexes that contain DNA, even if the DNA is not bacterial, but rather is host DNA, that normally would not trigger immune activation through TLR9. It was previously hypothesized that CpG DNA contributed to the pathogenesis of SLE, and that chloroquine and chloroquine-like compounds would be useful in preventing SLE. Krieg A M (1995) J Clin Immunol 15:284-92. It now appears more likely that CpG DNA does not need to be the trigger for lupus, but rather that any self DNA (i.e., CpG DNA or non-CpG DNA) can trigger this if the DNA is present as part of an immune complex.

The instant invention in one aspect provides a method for inhibiting immune activation. The method according to this aspect of the invention involves contacting a TLR9-expressing cell with an inhibitory nucleic acid and a small molecule antagonist of immunostimulatory CpG nucleic acids, in an effective amount to inhibit activation of the TLR9-expressing cell by a nucleic acid-containing immune complex.

As used herein, a “TLR9-expressing cell” refers to a cell that naturally or artificially expresses a functional TLR9 polypeptide or derivative thereof. The native, full-length amino acid sequences of human and murine TLR9 are publicly available in GenBank, for example as accession numbers AAF72189 and AAK29625, respectively. The functional TLR9 polypeptide or derivative thereof includes mutants, allelic variants, orthologs, fusion proteins, conjugates, etc., provided the TLR9 polypeptide or derivative thereof is capable of engaging an intracellular signaling pathway such as that includes MyD88, IRAK, and/or TRAF6 in response to bacterial or CpG DNA. For a recent review of TLR9 signaling, see Wagner H (2002) Curr Opin Microbiol 5:62-9. Cells that naturally express TLR9 include professional antigen-presenting cells, e.g., B cells, dendritic cells including plasmacytoid dendritic cells (pDCs), and macrophages. Cells that artificially express TLR9 include any cell into which has been introduced a nucleic acid sequence which encodes a TLR9 polypeptide, operatively linked to a gene expression sequence. Such nucleic acid sequences can include, for example, GenBank accession numbers AF259262 or AF245704 (human TLR9) and AF348140 (murine TLR9). The TLR9 polypeptide can be expressed constitutively or inducibly. Cells that express TLR9, either naturally or artificially, specifically include but are not limited to human cells.

In one embodiment the cell naturally expresses functional TLR9 and is an isolated cell from human multiple myeloma cell line RPMI 8226 (ATCC CCL-155). This cell line was established from the peripheral blood of a 61 year old man at the time of diagnosis of multiple myeloma (IgG lambda type). Matsuoka Y et al. (1967) Proc Soc Exp Biol Med 125:1246-50. RPMI 8226 was previously reported as responsive to CpG nucleic acids as evidenced by the induction of IL-6 protein and IL-12 p40 mRNA. Takeshita F et al. (2000) Eur J Immunol 30:108-16; Takeshita F et al. (2000) Eur J Immunol 30:1967-76. Takeshita et al. used the cell line solely to study promoter constructs in order to identify transcription factor binding sites important for CpG nucleic acid signaling. It is now known that RPMI 8226 cells secrete a number of other chemokines and cytokines including IL-8, IL-10 and IP-10 in response to immunostimulatory nucleic acids. Because this cell line expresses TLR9, through which immunostimulatory nucleic acids such as for example CpG nucleic acids mediate their effects, it is a suitable cell line for use in the methods of the invention relating to CpG nucleic acids as reference and test compounds, as well as to other TLR9 ligands.

Similar to peripheral blood mononuclear cells (PBMCs), the RPMI 8226 cell line has been observed to upregulate expression of cell surface markers such as CD71, CD86 and HLA-DR in response to CpG nucleic acid exposure. This has been observed by flow cytometric analysis of the cell line. Accordingly, the methods provided herein can be structured to use appropriately selected cell surface marker expression as a readout, in addition to or in place of chemokine or cytokine production or other readouts described elsewhere herein.

The RPMI cell line can be used in unmodified form or in a modified form. In one embodiment, the RPMI 8226 cell is transfected with a reporter construct. In one embodiment, the cell is stably transfected with the reporter construct. The reporter construct generally includes a promoter, a coding sequence and a polyadenylation signal. The coding sequence can include a reporter sequence selected from the group consisting of an enzyme (e.g., luciferase, alkaline phosphatase, beta-galactosidase, chloramphenicol acetyltransferase (CAT), secreted alkaline phosphatase, etc.), a bioluminescence marker (e.g., green fluorescent protein (GFP, U.S. Pat. No. 5,491,084), etc.), a surface-expressed molecule (e.g., CD25), a secreted molecule (e.g., IL-8, IL-12 p40, TNF-α, etc.), and other detectable protein products known to those of skill in the art. In one embodiment, the coding sequence encodes a protein having a level or an activity that is quantifiable.

In certain embodiments a functional TLR9 is artificially expressed (including over-expressed) by a cell, for example by introduction into the cell of an expression vector bearing a coding sequence for the functional TLR9 wherein the coding sequence is operably linked to a gene expression sequence. As used herein, a coding sequence and a gene expression sequence are said to be operably linked when they are covalently linked in such a way as to place the expression or transcription and/or translation of the coding sequence under the influence or control of the gene expression sequence. Two DNA sequences are said to be operably linked if induction of a promoter in the 5′ gene expression sequence results in the transcription of the coding sequence and if the nature of the linkage between the two DNA sequences does not (1) result in the introduction of a frame-shift mutation, (2) interfere with the ability of the promoter region to direct the transcription of the coding sequence, or (3) interfere with the ability of the corresponding RNA transcript to be translated into a protein. Thus, a gene expression sequence would be operably linked to a coding sequence if the gene expression sequence were capable of effecting transcription of that coding sequence such that the resulting transcript is translated into the desired protein or polypeptide.

In some embodiments a coding sequence refers to a nucleic acid sequence coding for a functional TLR9. In some embodiments a coding sequence refers to a nucleic acid sequence coding for a reporter.

A cell that artificially expresses a functional TLR9 can be a cell that does not express the functional TLR9 but for the TLR9 expression vector. For example, human 293 fibroblasts (ATCC CRL-1573) do not express TLR9. As described in the examples below, such cells can be transiently or stably transfected with suitable expression vector (or vectors) so as to yield cells that do express TLR9. Alternatively, a cell that artificially expresses a functional TLR9 can be a cell that expresses the functional TLR9 at a significantly higher level with the TLR9 expression vector than it does without the TLR9 expression vector.

For use in the methods of the instant invention, a cell that artificially expresses a functional TLR9 is in one embodiment a stably transfected cell that expresses the functional TLR9. Such a cell can also be stably transfected with a suitable reporter construct.

The terms “nucleic acid” and “oligonucleotide” are used interchangeably to mean multiple nucleotides (i.e., molecules comprising a sugar (e.g., ribose or deoxyribose) linked to a phosphate group and to an exchangeable organic base, which is either a substituted pyrimidine (e.g., cytosine (C), thymine (T) or uracil (U)) or a substituted purine (e.g., adenine (A) or guanine (G)). As used herein, the term refers to ribonucleotides as well as oligodeoxyribonucleotides (ODN). The terms “nucleic acid” and “oligonucleotide” shall also include polynucleosides (i.e., a polynucleotide minus the phosphate) and any other organic base containing polymer. Nucleic acid molecules can be obtained from existing nucleic acid sources (e.g., genomic or cDNA), but are preferably synthetic (e.g., produced by oligonucleotide synthesis).

The terms “nucleic acid” and “oligonucleotide” also encompass nucleic acids or oligonucleotides with non-nucleotide spacers, or substitutions or modifications, such as in the bases and/or sugars. For example, they include nucleic acids having backbone sugars that are covalently attached to low molecular weight organic groups other than a hydroxyl group at the 3′ position and other than a phosphate group at the 5′ position. Thus modified nucleic acids may include a 2′-O-alkylated ribose group. In addition, modified nucleic acids can include sugars such as arabinose instead of ribose. Thus the nucleic acids can be heterogeneous in backbone composition thereby containing any possible combination of polymer units linked together such as peptide-nucleic acids (which have an amino acid backbone with nucleic acid bases). Oligonucleotides can be made in linear or branched forms, or as multimers linked at one end (e.g., at the 3′ end) to a support such as a bead, nanoparticle, cationic polypeptide (e.g., poly-arginine), or cationic microparticle such as polylactide co-glycolide.

Nucleic acids also include substituted purines and pyrimidines such as C-5 propyne modified bases. Wagner R W et al. (1996) Nat Biotechnol 14:840-4. Purines and pyrimidines include but are not limited to adenine, cytosine, guanine, thymidine, 5-methylcytosine, 2-aminopurine, 2-amino-6-chloropurine, 2,6-diaminopurine, hypoxanthine, and other naturally and non-naturally occurring nucleobases, substituted and unsubstituted aromatic moieties. Other such modifications are well known to those of skill in the art.

An “inhibitory nucleic acid” as used herein refers to a nucleic acid that contains a specific sequence found to inhibit an immune response. In one embodiment an inhibitory nucleic acid is an inhibitory ODN. An “inhibitory ODN” as used herein refers to an inhibitory nucleic acid that is an oligodeoxynucleotide. Inhibitory ODN are generally up to about 100 nucleotides long and more typically are about 8-40 nucleotides long. More specifically, the inhibitory ODN inhibit apoptosis protection and cell-cycle entry induced by stimulatory ODN, but not that induced by lipopolysaccharide, anti-CD40, or anti-IgM plus IL-4. ODN-driven up-regulation of cyclin D₂, c-Myc, c-Fos, c-Jun and Bcl_{XL} and down-regulation of cyclin kinase inhibitor p27^{kip1} are all blocked by inhibitory ODN. Interference with uptake of immunostimulatory nucleic acids does not account for their inhibitory effects. At least partial inhibition of stimulatory effects of immunostimulatory nucleic acids occurs even if contact with inhibitory ODN is delayed for several hours following contact with immunostimulatory nucleic acid. Stunz L L et al. (2002) Eur J Immunol 32:1212-22. The specific sequences found to inhibit an immune response are referred to as “inhibitory motifs”. The inhibitory oligonucleotides of the invention contain at least one inhibitory motif. In one embodiment the inhibitory oligonucleotides are not antisense oligonucleotides.

The inhibitory motif can be a poly G motif. In various embodiments the poly G motif is GGGG, N₁GGGN₂GGGN₃ (SEQ ID NO:20), wherein N₁, N₂, and N₃ are each independently any nucleic acid sequence comprising 0-20 nucleotides, a sequence of 5 nucleotides in which at least 4 nucleotides are G, a sequence of 7 nucleotides in which at least 5 nucleotides are G, or a sequence of 8 nucleotides in which at least 6 nucleotides are G. Formation of G tetrads may not be required for activity of the inhibitory motif, so modified G's such as 7-deazaguanosine, can be used in place of G. In another embodiment the inhibitory motif can be a CCGG quadmer or more than one CCG or CGG trimer.

For purposes of this invention, certain inhibitory nucleic acid sequences are those that contain poly G motifs. In one embodiment the inhibitory nucleic acid will be stabilized in some way against degradation. In one embodiment the inhibitory nucleic acid will have a phosphorothioate backbone. Examples of inhibitory ODN include the following:

| ODN | Sequence (5′→3′) | SEQ ID NO: |
|---|---|---|
| 1483 | GTGCCGGGGTCTCCGGGC | 1 |
| 1484 | GCTGTGGGGCGGCTCCTG | 2 |
| 1628 | GGGGTCAACGTTGAGGGGGG | 3 |
| 1762 | GGGGAGGGT | 4 |
| 1763 | GGGGAGGGG | 5 |
| 1824 | CACGTTGAGGGGCAT | 6 |
| 2088 | TCCTGGCGGGGAAGT | 7 |
| 2114 | TCCTGGAGGGGAAGT | 8 |
| 2327 | GGCTCCGGGGAGGGAATTTTTGTCTAT | 9 |
| 2338 | TCCTGCCGGGGAAGT | 10 |
| 2339 | TCCTGCAGGGGAAGT | 11 |
| 2340 | TCCTGAAGGGGAAGT | 12 |
| 2341 | TCCTGGCGGGCAAGT | 13 |
| 2342 | TCCTGGCGGGTAAGT | 14 |
| 2343 | TCCTGGCGGGAAAGT | 15 |
| 2344 | TCCGGGCGGGGAAGT | 16 |
| 2345 | TCGGGGCGGGGAAGT | 17 |
| 2346 | TCCCGGCGGGGAAGT | 18 |
| 2347 | GGGGGACGTTGGGGG | 19 |

As used herein, immunostimulatory nucleic acids are nucleic acids that contain specific sequences found to elicit an immune response. These specific sequences are referred to as “immunostimulatory motifs”. The immunostimulatory nucleic acids, including immunostimulatory ODN, of the invention include at least one immunostimulatory motif.

In some embodiments of the invention the immunostimulatory nucleic acids include immunostimulatory motifs which are “CpG dinucleotides”. The CpG dinucleotides can be methylated or unmethylated. An immunostimulatory nucleic acid containing at least one unmethylated CpG dinucleotide is a nucleic acid molecule which contains an unmethylated cytosine-guanine dinucleotide sequence (i.e., DNA containing an unmethylated 5′ cytidine followed by 3′ guanosine and linked by a phosphate bond) and activates the immune system. Such immunostimulatory nucleic acids are referred to herein as CpG nucleic acids. The CpG dinucleotide can occur in the context of certain flanking nucleotides that taken together constitute a CpG motif. In one embodiment the CpG nucleic acid is DNA. CpG nucleic acids which are oligodeoxynucleotides are referred to as CpG ODN. Examples of CpG nucleic acids include, without limitation, those disclosed in U.S. Pat. Nos. 6,194,388; 6,207,646; 6,214,806; 6,218,371; 6,239,116; and 6,339,068.

An immunostimulatory nucleic acid containing at least one methylated CpG dinucleotide is a nucleic acid which contains a methylated cytosine-guanine dinucleotide sequence (i.e., a methylated 5′ cytidine followed by a 3′ guanosine and linked by a phosphate bond) and activates the immune system. In other embodiments the immunostimulatory oligonucleotides are free of CpG dinucleotides. The invention, therefore, also encompasses nucleic acids with other types of immunostimulatory motifs, which can be methylated or unmethylated, and the immunostimulatory oligonucleotides of the invention can, therefore, include a combination of methylated and unmethylated immunostimulatory or non-immunostimulatory motifs.

In one embodiment the inhibitory nucleic acid has a stabilized backbone, i.e., the inhibitory nucleic acid is a stabilized nucleic acid molecule. A “stabilized backbone” as used herein in reference to a nucleic acid molecule shall mean a backbone that is relatively resistant to in vivo degradation (e.g., via an exo- or endo-nuclease). A “stabilized nucleic acid molecule” shall mean a nucleic acid molecule that is relatively resistant to in vivo degradation (e.g., via an exo- or endo-nuclease). Stabilization can be a function of length or secondary structure. For example, unmethylated CpG-containing nucleic acid molecules that are tens to hundreds of kilobases long are relatively resistant to in vivo degradation. For shorter immunostimulatory nucleic acid molecules, secondary structure can stabilize and increase their effect. For example, if the 3′ end of a nucleic acid molecule has self-complementarity to an upstream region, so that it can fold back and form a sort of stem loop structure, then the nucleic acid molecule becomes stabilized and therefore exhibits more activity.

Certain stabilized nucleic acid molecules of the instant invention have a modified backbone. In one embodiment stabilized nucleic acid molecules include a phosphate backbone modification. The phosphate backbone modification can involve substitution of at least one of the phosphate oxygens with sulfur, such that the resulting stabilized nucleic acid molecules are phosphorothioate- or phosphorodithioate-modified nucleic acid molecules. As used herein, a “phosphorothioate backbone” refers to a backbone including at least one phosphorothioate intemucleotide linkage. In one embodiment every intemucleotide linkage is a phosphorothioate linkage. If chirality of the phosphorothioate linkage is specified, in one embodiment the configuration is Sp for improved nuclease resistance at the 5′ and 3′ ends especially. In other embodiments the backbone includes phosphorothioate linkages between some but not all nucleotides. More particularly, in one embodiment the phosphate backbone modification occurs at the 5′ end of the nucleic acid, for example, at the first two nucleotides of the 5′ end of the nucleic acid. In one embodiment the phosphate backbone modification can occur at the 3′ end of the nucleic acid, for example, at the last five nucleotides of the 3′ end of the nucleic acid. In one embodiment the phosphate backbone modification occurs both at the 5′ end of the nucleic acid, for example, at the first two nucleotides of the 5′ end of the nucleic acid, and at the 3′ end of the nucleic acid, for example, at the last five nucleotides of the 3′ end of the nucleic acid.

Other stabilized nucleic acid molecules include: nonionic DNA analogs, such as alkyl- and aryl-phosphonates (in which the charged phosphonate oxygen is replaced by an alkyl or aryl group), phosphodiester and alkylphosphotriesters, in which the charged oxygen moiety is alkylated. Nucleic acid molecules which contain a diol, such as tetraethyleneglycol or hexaethyleneglycol, at either or both termini have also been shown to be substantially resistant to nuclease degradation.

In some embodiments the immunostimulatory nucleic acid is a stabilized nucleic acid molecule.

For use in the instant invention, nucleic acids can be synthesized de novo using any of a number of procedures well known in the art. For example, the nucleic acids can be synthesized de novo using the β-cyanoethyl phosphoramidite method (Beaucage S L et al. (1981) Tetrahedron Lett 22:1859-62) or the nucleoside H-phosphonate method (Garegg P J et al. (1986) Tetrahedron Lett 27:4051-4; Froehler B C et al. (1986) Nucl Acid Res 14:5399-407; Garegg P J et al. (1986) Tetrahedron Lett 27:4055-8; Gaffney B L et al. (1988) Tetrahedron Lett 29:2619-22). These chemistries can be performed by a variety of automated oligonucleotide synthesizers available in the market. Alternatively, oligonucleotides can be prepared from existing nucleic acid sequences (e.g., genomic or cDNA) using known techniques, such as those employing restriction enzymes, exonucleases or endonucleases.

For use in vivo, nucleic acids are preferably relatively resistant to degradation (e.g., via endo- and exo-nucleases). A preferred stabilized nucleic acid has at least a partial phosphorothioate modified backbone. Phosphorothioates can be synthesized using automated techniques employing either phosphoramidate or H-phosphonate chemistries. Aryl- and alkyl-phosphonates can be made, e.g., as described in U.S. Pat. No. 4,469,863; and alkylphosphotriesters (in which the charged oxygen moiety is alkylated as described in U.S. Pat. No. 5,023,243 and European Patent No. 092,574) can be prepared by automated solid phase synthesis using commercially available reagents. Methods for making other DNA backbone modifications and substitutions have been described. Uhlmann E et al. (1990) Chem Rev 90:543-84; Goodchild J (1990) Bioconjugate Chem 1:165-87.

The methods of the invention involve inhibition of activation of a TLR9-expressing cell by a nucleic acid-containing immune complex. As used herein, a “nucleic acid-containing immune complex” is a conjugate formed between an antibody and a nucleic acid. The complex need not involve a covalent linkage between the antibody and nucleic acid components. In one embodiment the antibody is an intact antibody, but it can be any fragment of an antibody that forms a conjugate with the nucleic acid molecule, either directly or indirectly. The antibody can be found in nature, or it can be polyclonal, monoclonal, chimeric, humanized, polyspecific, conjugated with yet another compound, or otherwise derived from an antibody found in nature or derived in vitro using methods well known in the art. Indirect binding of a nucleic acid by an antibody can occur where the antibody binds to another molecule associated with the nucleic acid, e.g., a histone. The complex can include a plurality of antibodies, and it independently can include a plurality of nucleic acid molecules. For example, IgG antibodies found in nature are bivalent, i.e., each IgG can bind two antigens; the two antigens can be on separate molecules or they can be separate parts of a single molecule or single complex.

In one embodiment the nucleic acid-containing immune complex includes a CpG nucleic acid.

In one embodiment the nucleic acid-containing immune complex includes a bacterial nucleic acid. As used herein, “bacterial nucleic acid” refers to DNA or RNA originating from bacteria. In one embodiment the nucleic acid-containing immune complex includes a viral nucleic acid. As used herein, “viral nucleic acid” refers to DNA or RNA originating from a virus. In one embodiment the nucleic acid-containing immune complex includes a host nucleic acid. As used herein, “host nucleic acid” refers to DNA or RNA originating from a vertebrate host. Bacterial DNA characteristically has a higher CpG content than vertebrate DNA. Other non-host nucleic acids are also contemplated by the invention, including DNA or RNA originating from viral, retroviral, fungal, and parasitic sources. In some cases these non-host nucleic acids have a higher CpG content than vertebrate DNA; in some cases these non-host nucleic acids have a CpG content similar to or even lower than vertebrate DNA. Some of these nucleic acids contain immune inhibitory motifs and will inhibit immune stimulation by conventional CpG DNA.

The methods of the invention include contacting a TLR9-expressing cell with a small molecule antagonist of immunostimulatory CpG nucleic acids. As used herein, a “small molecule antagonist of immunostimulatory CpG nucleic acids” is any small molecule compound, other than an inhibitory oligonucleotide, that inhibits CpG nucleic acid-related immunostimulation. It is believed, for example, that such antagonist molecules can interfere with TLR9-mediated signalling, albeit through an as-yet undefined mechanism. In one embodiment the small molecule antagonist of immunostimulatory CpG nucleic acids is a compound that selectively inhibits endosomal acidification or that selectively inhibits endosomal maturation. Such compounds include chloroquine, hydroxychloroquine, quinacrine, and derivatives and analogs of these molecules as described herein and as described in U.S. Pat. No. 6,221,882, published PCT application PCT/US00/16723 (WO 00/76982), and in Strekowski L et al. (1999) Bioorg Med Chem Lett 9:1819-24, Strekowski L et al. (2003) J Med Chem 46:1242-9, and Strekowski L et al. (2003) Bioorg Med Chem 11:1079-85. These small molecule antagonists of immunostimulatory CpG nucleic acids can be useful in the instant invention when used at concentrations substantially below concentrations at which they exhibit antimalarial activity.

In selecting a small molecule antagonist of immunostimulatory CpG nucleic acids, it is generally advantageous to select one with low EC₅₀, i.e., with a low concentration required for half-maximal inhibition of CpG DNA induced cell stimulation index. Hydroxychloroquine (PLAQUENIL®, Sanofi-Synthelabo) is reported to have an EC₅₀ of about 300-400 nM. In certain embodiments the EC₅₀ is less than about 100 nM. In certain embodiments the EC₅₀ is less than about 10 nM. In certain embodiments the EC₅₀ is less than about 1 nM.

An inhibitor of endosomal acidification/maturation can also be bafilomycin A, monensin, concanamycin B, or ammonium chloride, all of which are commercially available from Sigma-Aldrich. Additional inhibitors of endosomal acidification/maturation or trafficking, such as monodansylcadaverine and inhibitors of Rab 5, which block homotypic fusion of early endosomes (Ahmad-Nejad P et al. (2002) Eur J Immunol 32:1958-68), are also contemplated by the instant invention.

In one embodiment the small molecule antagonist of immunostimulatory CpG nucleic acids is chosen from 4-aminoquinoline compounds having the structural Formula 1:

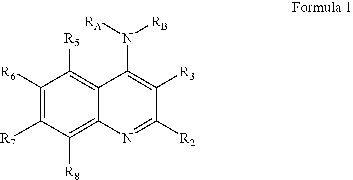

wherein R_{A} is a hydrogen atom, a lower alkyl group, or linked to R_{B} by a substituted or unsubstituted alkyl chain; R_{B} is a hydrogen atom, an alicyclic group, an alkyl secondary, tertiary or quaternary amine, or an alkenyl secondary, tertiary or quaternary amine; R₂ is a hydrogen atom, a lower alkyl group, an aryl group, a heteroaromatic group, or a lower alkenyl group substituted with an aryl group; R₃ is a hydrogen atom, a lower alkyl group, or an aromatic group; R₅ is a hydrogen atom, a lower alkyl group, or a halogen atom; R₆ is a hydrogen atom, a lower alkyl group, a lower alkoxy group, an aryloxy group, an aryl group, an amino group, or a thioether group; R₇ is a hydrogen atom, a lower alkyl group, a lower alkoxy group, an aryloxy group, a haloalkyl group, or a halogen atom; and R₈ is a hydrogen group, or a lower alkoxy group, and pharmaceutically acceptable salts thereof, with the proviso that if R₇ is a halogen, then at least one of R₂, R₃, R₅, R₆ or R₈ is non-hydrogen and R_{B} is not 4-[N,N-dialkyl-n-pentylamine] or 4-[N-alkyl-N-hydroxyalkyl-n-pentylamine].

According to this embodiment, R_{A} can be a hydrogen atom, a lower alkyl group, or can be further linked to R_{B} by an alkyl chain. If R_{A} is a lower alkyl group, it is in certain embodiments methyl or ethyl. If R_{A} is further linked to R_{B} by an alkyl chain (in addition to the depicted linkage by the nitrogen atom), the alkyl chain can be —(CH₂)ₙ—, where n is from 4 to 7, and is in certain embodiments 4 or 5.

The substituent R_{B} can be a hydrogen atom, an alicyclic group (such as a cyclopentyl, cyclohexyl or cycloheptyl group), an alkyl secondary, tertiary or quaternary amino group or alkenyl secondary, tertiary or quaternary amino group. For example, R_{B} can be an unsubstituted alkyl amine, such as 4-[pentyl-N,N-dialkyl amine], 4-[pentyl-N-alkyl amine], 4-[pentyl amine], 4-[butyl-N,N-dialkyl amine], 4-[butyl-N-alkyl amine], 4-[butyl amine], 2-[ethyl-N,N-dialkyl amine], 2-[ethyl-N-alkyl amine], 2-[ethyl amine], 3-[propyl-N,N-dialkyl amine], 3-[propyl-N-alkyl amine], and 7-[hepta-4-methyl-4-azaamine]. The N-substituents are generally lower alkyl, but can also include hydroxy-substituted lower alkyl, such as 2-hydroxyethyl. Unsaturated chains include 4-[pent-2-enyl-N,N-dialkyl amine] and 4-[pent-2-enyl-N-alkyl amine]. Longer amine-containing alkyl chains can also be utilized, and the nitrogen need not be located at the terminus of the substituent group. Cyclic amines can be included in the alkyl chain. For example, the alkyl chain can terminate in, or be interrupted by, a pyrrole ring, a piperazidyl ring, a piperidyl ring, or a morpholinyl ring, any of which may be further substituted with lower alkyl groups. In embodiments in which R_{B} is an unsubstituted amine-containing group, in certain embodiments R_{B} can be 2-[ethyl-N,N-dimetlhyl amine], 2-[ethyl-N-methyl amine], 4-[pentyl-N,N-diethyl amine], and 4-[butyl-N,N-diethyl amine].

The R_{B} amine-containing chains described above can be variously substituted. Substituents include lower alkyl groups, such as methyl, ethyl and propyl. Other useful R_{B} substituents include substituted or unsubstituted aryl groups, such as phenyl, anisyl, hydroxyphenyl, chlorophenyl, dichlorophenyl, fluorophenyl, naphthyl, thiophelnyl, which can be substituted at o-, m- or p-positions, or at 1- or 2-positions in case of naphthyl; heterocyclic groups, such as pyridyl, pyrrolyl, piperidyl, and piperazidyl; or halogen, such as chloro, bromo, and fluoro; and other substituents such as hydroxyl and alkoxyl. Further substitution of this amine-containing alkyl chain can include, for example, amide or ester linkages, ether or thioether linkages. The amine-containing alkyl chain can terminate with a substituent group such as a primary, secondary or tertiary amine, a hydroxy group, a thiol, a carboxylic acid, or an amide.

R_{B} can also be an alipolycyclic group, such as bicycloheptyl, bicyclooctyl, or adamantyl, and can be linked to any position of these groups. R_{B} can also be linked to R_{A} by an alkyl chain.

The substituent R₂ can be a hydrogen atom, a lower alkyl group, or an aryl group. R₂ can also be a heteroaromatic group, such as 2-, 3-, or 4-pyridyl, 1-, 2-, or 3-pyrrolyl, or an aryl substituted lower alkenyl group, such as trans-β-styryl and trans-β-[α,β-trans-dimethyl-p-chlorostyryl]. It is believed that bulky groups at R₂ contribute to the efficacy of the compounds of the invention. Thus, many of the embodiments include bulky groups at this position. Among the unsubstituted aryl groups useful as R₂ substituents include phenyl, 1- and 2-naphthyl, 1-, 2-, 3-, 4-, or 9-phenanthryl and the like. Among the substituted aromatic hydrocarbons include the above-mentioned aryl groups, substituted with lower alkyl groups such as methyl and ethyl; halogens, such as chlorine, fluorine and bromine; perfluoroalkyl groups such as trifluoromethyl and pentafluoroetlhyl; alkoxy groups, such as methoxy; aryloxy groups such as phenoxy; amine-containing substituents such as N-[N,N-dimethyl ethylenediamine], or 1-[4-methylpiperazine]. Any of the above groups can be present at o-, m-, or p-positions on a phenyl ring, or at any synthetically feasible position on another aryl system. Multiply substituted aryl rings are also possible.

In certain embodiments R₂ is 4-(N-methylpiperazino)phenyl.

The substituent R₃ can be a hydrogen atom, a lower alkyl group such as methyl or ethyl, or an aromatic group such as phenyl. In certain embodiments, the substituent R₃ is hydrogen or methyl.

The substituent R₅ can be a hydrogen atom, a lower alkyl group such as methyl or ethyl, or a halogen atom, such as chlorine, bromine or fluorine.

The substituent R₆ can be a hydrogen atom, a lower alkyl group such as methyl or ethyl, a lower alkoxy group such as methoxy or ethoxy, an aryloxy group such as phenoxy, an aryl group such as phenyl, an amine group such as N,N-dimethyl amino or N,N-diethylamino, or a thioether group such as phenylthioether or benzylthioether. It is found that bulky substituents on position 6 of the quinoline ring tend to enhance activity, so that many of the preferred embodiments include bulky groups at this position.

The substituent R₇ can be a hydrogen atom, a lower alkyl group such as methyl or ethyl, a lower alkoxy group such as methoxy or ethoxy, an aryloxy group such as phenoxy, a halogen atom such as chlorine, bromine or fluorine, or a lower haloalkyl group, namely a lower perfluoroalkyl group, such as trifluromethyl or pentafluoroethyl.

The substituent R₈ can be a hydrogen atom or a lower alkoxy group such as methoxy or ethoxy. Pharmaceutically acceptable salts of any of these compounds are also included in the invention. These salts include protonated or deprotonated atoms on the 4-aminoquinoline and counterions including potassium, sodium, chlorine, bromine, acetate and many other commonly recognized counterions.

If R₇ is a halogen, then at least one of R₂, R₃, R₅, R₆, or R₈ is not a hydrogen atom and R_{B} is not 4-[N,N-dialkyl-n-pentylamine] or 4-[N-alkyl-N-hydroxyalkyl-n-pentylamine].

In one embodiment the 4-aminoquinoline compound is N-[3-(dimethylamino)propyl]-2-[4-(N-methylpiperazino)phenyl]quinolin-4-amine (i.e., compound 50 of Strekowski L et al. (2003) J Med Chem 46:1242-9).

In one embodiment the 4-aminoquinoline compound is N-[2-(dimethylamino)ethyl]-2-[4-(N-methylpiperazino)phenyl]quinolin-4-amine (i.e., compound 47 of Strekowski L et al. (2003) J Med Chem 46:1242-9).

In one embodiment the 4-aminoquinoline compound is N-[4-(dimethylamino)butyl]-2-[4-(N-methylpiperazino)phenyl]quinolin-4-amine (i.e., compound 44 of Strekowski L et al. (2003) J Med Chem 46:1242-9).

The 4-aminoquinoline compounds can be linked together via a linker. The linker can be connected to each molecule at either the same position on the compound, or at different positions. Certain linked 4-aminoquinoline compounds are linked at the same position on each compound. In one embodiment the position for linkage between the individual 4-aminoquinoline compounds is at the 4-amino position, shown as R_{A} or R_{B} in the structure. The linker can be of a number of types. Certain linker types are alkyl chains, alkyl chains interrupted with nitrogen atoms, or alkyl chains interrupted with amide linkages. In certain embodiments the alkyl chains are at least two carbons in length and not more than twelve carbons in length. If the alkyl chain is interrupted by nitrogen atoms, in certain embodiments the overall length of the chain is between two and twelve atoms. Certain linker molecules include those of the formula —[(CH₂)ₙ₁N(R)(CH₂)ₙ₂]ₙ₃— where n1, n2 and n3 are independently between 1 and 5, and R is a hydrogen atom, a carbonyl group or a lower alkyl group. The linker can further include substituents to vary the hydrophobicity or hydrophilicity of the linked compound as a whole.

In one embodiment the 4-aminoquinoline compound is a phenyl-linked bis-4-aminoquinoline compound. In one embodiment the phenyl-linked bis-4-aminoquinoline compound is N,N′-Bis[4-[4-[2-(dimethylamino)ethyl]amino]quinolin-2-yl]phenyl]hexane-1,6-diamine (i.e., compound 16 of Strekowski L et al. (2003) Bioorg Med Chem 11:1079-85). In one embodiment the phenyl-linked bis-4-aminoquinoline compound is N,N′-Bis[4-[4-[2-(dimethylamino)ethyl]amino]quinolin-2-yl]phenyl]-4,9-dioxa-1,12-dodecanediamine (i.e., compound 19 of Strekowski L et al. (2003) Bioorg Med Chem 11:1079-85).

In one embodiment the small molecule antagonist of immunostimulatory CpG nucleic acids is chosen from 9-aminoacridine compounds having the structural Formula 2:

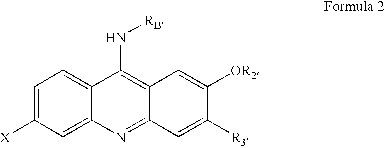

wherein R_{B′} is a hydrogen atom or an alkyl secondary, tertiary, or quaternary amino group; R_{2′} is a lower alkyl group; R_{3′} is a hydrogen atom or a lower alkoxy group; X is a halogen atom; and pharmaceutically acceptable salts thereof.

According to this embodiment, the substituent R_{B′} can be a hydrogen atom or an alkyl secondary, tertiary or quaternary amino group. For example, R_{B′} can be an unsubstituted alkyl amine, such as 4-[pentyl-N,N-dialkyl amine], 4-[pentyl-N-alkyl amine], 4-[pentyl amine], 4-[butyl-N,N-dialkyl amine], 4-[butyl-N-alkyl amine], 4-[butyl amine], 2-[ethyl-N,N-dialkyl amine], 2-[ethyl-N-alkyl amine], 2-[ethyl amine], 3-[propyl-N,N-dialkyl amine], 3-[propyl-N-alkyl amine], and 7-[hepta-4-methyl-4-azaamine]. The N-substituents are generally lower alkyl, but can also include hydroxy-substituted lower alkyl, such as 2-hydroxyethyl. Unsaturated chains include 4-[pent-2-enyl-N,N-dialkyl amine] and 4-[pent-2-enyl-N-alkyl amine]. Longer amine-containing alkyl chains are also possible, and the nitrogen need not be located at the terminus of the substituent group. Cyclic amines can be included in the alkyl chain. For example, the alkyl chain can terminate in, or be interrupted by, a pyrrole ring, a piperazidyl ring, a piperidyl ring, or a morpholinyl ring, any of which can be further substituted with lower alkyl groups. In embodiments in which R_{B′} is an unsubstituted amine-containing group, certain variants of R_{B′} are 2-[N,N-dimethyl ethylamine], 2-[N-methyl ethylamine], 4-[N,N-diethyl pentylamine], and 4-[N,N-diethyl butylamine]. Certain embodiments of the 9-aminoacridines useful in the invention have R_{B′} as 4-[4-aryl-N,N-dialkyl butylamine], 4-[4-heteroaromatic-N,N-dialkyl butylamine], 4-[4-aryl-N-alkyl butylamine], and 4-[4-heteroaromatic-N-alkyl butylamine]. Quaternary nitrogen-containing variants of these residues are also envisioned as useful in the invention.

The R_{B′} amine-containing chains described above can be variously substituted. Substituents include lower alkyl groups, such as methyl, ethyl and propyl. Other useful R_{B′} substituents include substituted or unsubstituted aryl groups, such as phenyl, anisyl, hydroxyphenyl, chlorophenyl, dichlorophenyl, fluorophenyl, naphthyl, thiophenyl, which can be substituted at o-, m- or p-positions, or at 1- or 2-positions in case of naphthyl; heterocyclic groups, such as pyridyl, pyrrolyl, piperidyl, and piperazidyl; or halogen, such as chloro, bromo, and fluoro; and other substituents such as hydroxyl, and alkoxyl. Further substitution of this amine-containing alkyl chain can comprise, for example, amide or ester linkages, ether or thioether linkages. The amine-containing alkyl chain can terminate with a substituent group such as a primary, secondary or tertiary amine, an hydroxy group, a thiol, a carboxylic acid, or an amide. Preferred embodiments have 4-[N,N-dialkyl pentylamine], 4-[4-aryl-N,N-dialkyl butylamine] or 4-[4-heteroaromatic-N,N-dialkyl butylamine] groups at this position, in which the aryl or heteroaromatic group is unsubstituted or substituted by halogen or alkoxy, and the N,N-dialkyl groups are N,N-diethyl groups.

The substituent R_{2′} is a lower alkyl group, such as methyl or ethyl. Certain embodiments have methyl at this position.

The substituent R_{3′} can be a hydrogen atom or a lower alkoxy group.

The substituent X is a halogen atom, such as chlorine, bromine or fluorine. Certain embodiments have chlorine at this position.

The 9-aminoacridine compounds can be linked together via a linker. The linker can be connected to each molecule at either the same position on the compound, or on different positions. Certain linked 9-aminoacridine compounds are linked at the same position on each compound. In one embodiment the position for linkage between the individual 9-aminoacridine compounds is at the 9-amino position, shown as R_{A′} or R_{B′} in structural Formula 2. The linker can be of a number of types. Certain linker types are alkyl chains, alkyl chains interrupted with nitrogen atoms, or alkyl chains interrupted with amide linkages. In certain embodiments the alkyl chains are at least two carbons in length and not more than twelve carbons in length. If the alkyl chain is interrupted by nitrogen atoms, in certain embodiments the overall length of the chain is between two and twelve atoms. Certain linker molecules include those of the formula —[(CH₂)ₙ₁N(R)(CH₂)ₙ₂]ₙ₃— where n1, n2 and n3 are independently between 1 and 5, and R is a hydrogen atom, a carbonyl group or a lower alkyl group.

Further useful compounds include 4-aminoquinoline compounds and 9-aminoacridine compounds linked together by a linker. Certain linked 4-aminoquinoline/9-aminoacridine compounds are linked at analogous positions on each compound. In one embodiment the preferred position for linkage for the 9-aminoacridine compounds is at the 9-amino position, shown as R_{A′} or R_{B′} in the structure and the linker is further linked to the 4-amino position of the 4-aminoquinoline compound. The linker can be of a number of types. Certain linker types are those discussed above in connection with the 4-aminoquinoline and 9-aminoacridine compounds.

In some embodiments the small molecule antagonist of immunostimulatory CpG nucleic acids is a compound of Formula 3, wherein Ar is selected from 2-naphthyl, 3-phenanthryl, 4-MePh, and trans-CH═CHPh. Strekowski L et al. (1999) Bioorg Med Chem Lett 9:1819-24. In one embodiment Ar is 2-naphthyl.

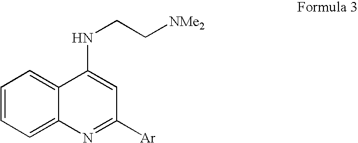

In some embodiments the small molecule antagonist of immunostimulatory CpG nucleic acids is a compound of Formula 4, wherein n is an integer between 3 and 6, inclusive, and R is selected from p-tolyl or 2-naphthyl when n is 3, 2-naphthyl when n is 4, and 2-naphthyl when n is 6. Strekowski L et al. (1999) Bioorg Med Chem Lett 9:1819-24.

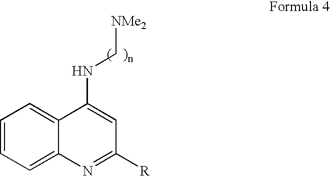

In some embodiments the small molecule antagonist of immunostimulatory CpG nucleic acids is a compound of Formula 5, wherein R′ is (CH₂)₃N(CH₂CH₂)₂N(CH₂)₃NHC(O)(CH₂)₃OH. Strekowski L et al. (1999) Bioorg Med Chem Lett 9:1819-24.

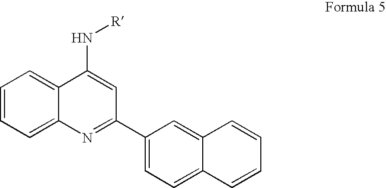

In some embodiments the small molecule antagonist of immunostimulatory CpG nucleic acids is a compound of Formula 6, wherein R_{1″} is selected from morpholinomethyl, piperidinomethyl, pyrrolidinomethyl, and N-methylpiperazinomethyl. In one embodiment of Formula 6, R_{1″} is N-methylpiperazinomethyl. Strekowski L et al. (1999) Bioorg Med Chem Lett 9:1819-24.

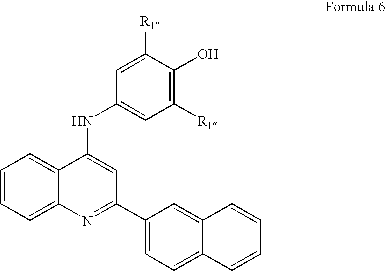

As used herein, a “subject having an autoimmune disease” is a subject with a recognizable sign or symptom of an existing autoimmune disease in the subject. A “subject at risk of developing an autoimmune disease” is a subject with a genetic or other predisposition toward developing an autoimmune disease. Such predisposition can include, for example, recognized MHC antigens associated with specific autoimmune disease. For example, HLA-B27 is reported to be associated with ankylosing spondylitis, Reiter's syndrome, psoriatic arthritis, and juvenile rheumatoid arthritis; HLA-DR₄ is reported to be associated with type 1 diabetes mellitus and SLE; and HLA-DR_{4/}Dw4, HLA-DR_{4/}Dw14, HLA-DR_{4/}Dw15, and others are reported to be associated with RA.

Autoimmune diseases include, without limitation, Hashimoto's thyroiditis, Graves' disease, Type I and Type II autoimmune polyglandular syndromes, type 1 (insulin-dependent) diabetes mellitus, immune-mediated infertility, autoimmune Addison's disease, pemphigus vulgaris, pemphigus foliaceus, paraneoplastic pemphigus, bullus pemphigoid, dermatitis herpetiformis, linear IgA disease, epidermolysis bullosa acquisita, autoimmune alopecia, erythema nodosa, pemphigoid gestationis, cicatricial pemphigoid, chronic bullous disease of childhood, autoimmune hemolytic anemia, autoimmune thrombocytopenic purpura, autoimmune neutropenia, myasthenia gravis, Eaton-Lambert myasthenic syndrome, stiff-man syndrome, acute disseminated encephalomyelitis, multiple sclerosis, Guillain-Barré syndrome, chronic inflammatory demyelinating polyradiculoneuropathy, multifocal motor neuropathy with conduction block, chronic neuropathy with monoclonal gammopathy, opsonoclonus-myoclonus syndrome, cerebellar degeneration, encephalomyelitis, retinopathy, autoimmune chronic active hepatitis, primary biliary sclerosis, sclerosing cholangitis, gluten-sensitive enteropathy, pernicious anemia, inflammatory bowel disease, SLE, RA, systemic sclerosis (scleroderma), ankylosing spondylitis, reactive arthritides, polymyositis/dermatomyositis, Sjögren's syndrome, mixed connective tissue disease, Bechet's syndrome, psoriasis, polyarteritis nodosa, allergic anguitis and granulomatosis (Churg-Strauss disease), polyangiitis overlap syndrome, hypersensitivity vasculitis, Wegener's granulomatosis, temporal arteritis, Takayasu's arteritis, Kawasaki's disease, isolated vasculitis of the central nervous system, thromboangiutis obliterans, sarcoidosis, GvHD, glomerulonephritis, and cryopathies. These conditions are well known in the medical arts and are described, for example, in Harrison 's Principles of Internal Medicine, 14^{th} ed., Fauci A S et al., eds., New York: McGraw-Hill, 1998.

Autoimmune diseases in which there are immune complexes which contain nucleic acids such as DNA or RNA are most notably RA and SLE. Additional inflammatory and autoimmune diseases in which there are also believed to be immune complexes which contain nucleic acids include: inflammatory bowel disease (IBD) including Crohn's disease and ulcerative colitis; Sjögren's syndrome; multiple sclerosis (MS) and experimental allergic encephalomyelitis (EAE), an animal model for MS; type 1 diabetes mellitus; certain viral infections, including in particular those associated with such viruses as hepatitis B virus (HBV) and hepatitis C virus (HCV); graft-versus-host disease (GvHD); and paraneoplastic autoimmune syndromes that can be associated with some malignancies, including, e.g., small cell lung cancer and breast cancer.

As used herein, an “effective amount” of a compound refers generally to an amount of that compound necessary or sufficient to achieve a desired biologic effect. Administration of an effective amount can involve administering a single dose or more than one dose.

In one aspect of the invention, a TLR₉-expressing cell is contacted with an inhibitory nucleic acid and a small molecule antagonist of immunostimulatory CpG nucleic acids, in an effective amount to inhibit activation of the TLR₉-expressing cell by a nucleic acid-containing immune complex. Thus an effective amount of a compound, or of a combination of compounds, to inhibit activation of a TLR₉-expressing cell is the amount necessary or sufficient to inhibit at least one manifestation of activation of the TLR₉-expressing cell under similar conditions in the absence of the compound or combination of compounds. A manifestation of activation of the TLR₉-expressing cell can be any one or combination of cellular proliferation, intracellular signaling, intercellular signaling, or expression or secretion of a soluble polypeptide or soluble polypeptide-containing product of the TLR₉-expressing cell in the presence of, or in response to, a suitable stimulus.

Activation of a TLR₉-expressing cell can be measured by any method suitable for measuring cellular proliferation, intracellular signaling, intercellular signaling, or expression or secretion of a soluble polypeptide or soluble polypeptide-containing product of the TLR₉-expressing cell. Such methods can include, without limitation, tritiated thymidine uptake, enzyme-linked immunosorbent assay (ELISA), fluorescence-activated cell sorting (FACS), and reporter construct assays, e.g., NF-κB-luciferase.

In another aspect of the invention, a subject having or at risk of developing an autoimmune disease is administered an inhibitory nucleic acid and a small molecule antagonist of immunostimulatory CpG nucleic acids, in an effective amount to treat or prevent the autoimmune disease. Thus an effective amount of a compound, or of a combination of compounds, to treat or prevent the autoimmune disease is the amount necessary or sufficient to treat or prevent at least one manifestation of the autoimmune disease. As used herein, the term “treat” refers to eliminating, halting or reducing progression of, a measurable sign or symptom of a disease or disorder of a subject.

The frequency and mode of administration of the compounds will depend on the nature of the disease being treated, but will require that the compounds be delivered at effective concentrations, directly or indirectly, to the tissue in which the immune activation is occurring. These tissues include primarily the lymph nodes and spleen, but also the bone marrow, liver, and blood.

Small molecule antagonists of immunostimulatory CpG nucleic acids such as chloroquine and related chloroquine-like compounds described herein can be administered by any suitable route of administration, and typically by mouth, at doses that will provide a steady state tissue concentration of from 10 pg/mL to 100 ng/mL, depending on the potency of the particular inhibitor compound. (See, e.g., the range of effective concentrations provided in Strekowski L et al. (1999) Bioorg Med Chem Lett 9:1819-24.) These compounds typically can be administered one or more times daily, but they can be administered every other day, or less frequently, depending on their pharmacokinetics.

The inhibitory nucleic acid can be administered by any suitable route of administration and are effective at less than equimolar concentrations relative to the stimulatory nucleic acid. The inhibitory nucleic acid will typically be delivered via a route of administration effective to achieve systemic distribution. Such routes of administration include, but are not limited to, intravenous, intramuscular, subcutaneous, oral, enteral, mucosal, intranasal, intrapulmonary, and intraperitoneal. In certain embodiments doses of the inhibitory nucleic acid typically range from 1 μg/kg to 10 mg/kg, and in certain embodiments doses of the inhibitory nucleic acid range from 100 μg/kg to 1 mg/kg. Dosing typically will involve administration on a daily to weekly schedule.

The inhibitory nucleic acid and the small molecule antagonist of immunostimulatory CpG nucleic acids thus can be administered by the same or different routes of administration. When the inhibitory nucleic acid and the small molecule antagonist of immunostimulatory CpG nucleic acids are administered by the same route of administration, the two components can be provided in a single formulation or in separate formulations.

The inhibitory nucleic acid and the small molecule antagonist of immunostimulatory CpG nucleic acids also can be administered or contacted with a TLR₉-expressing cell at the same time or at different times. In one embodiment the inhibitory nucleic acid is administered or contacted with the TLR₉-expressing cell before the small molecule antagonist of immunostimulatory CpG nucleic acids is administered or contacted with the TLR₉-expressing cell. In another embodiment the inhibitory nucleic acid is administered or contacted with the TLR₉-expressing cell after the small molecule antagonist of immunostimulatory CpG nucleic acids is administered or contacted with the TLR₉-expressing cell. In various embodiments the delay between the administering or contacting of the inhibitory nucleic acid and the administering or contacting of small molecule antagonist of immunostimulatory CpG nucleic acids is at least several hours.

The inhibitory nucleic acids can be directly administered to the subject or they can be administered in conjunction with a nucleic acid delivery complex. A nucleic acid delivery complex shall mean a nucleic acid molecule associated with (e.g., ionically or covalently bound to or encapsulated within) a targeting means (e.g., a molecule that results in higher affinity binding to target cell (e.g., B cell surfaces and/or increased cellular uptake by target cells). Examples of nucleic acid delivery complexes include nucleic acids associated with a sterol (e.g., cholesterol), a lipid (e.g., a cationic lipid, virosome or liposome), or a target cell specific binding agent (e.g., a ligand recognized by target cell specific receptor). Certain complexes can be sufficiently stable in vivo to prevent significant uncoupling prior to internalization by the target cell. However, the complex can be cleavable under appropriate conditions within the cell so that the nucleic acid is released in a functional form.

Delivery vehicles or delivery devices for delivering nucleic acids to surfaces have been described. The inhibitory oligonucleotide and/or other therapeutics can be administered alone (e.g., in saline or buffer) or using any delivery vehicles known in the art. For instance the following delivery vehicles have been described: cochleates (Gould-Fogerite et al., 1994, 1996); emulsomes (Vancott et al., 1998; Lowell et al., 1997); ISCOMs (Mowat et al., 1993; Carlsson et al., 1991; Hu et al., 1998; Morein et al., 1999); liposomes (Childers et al., 1999; Michalek et al., 1989, 1992; de Haan 1995a, 1995b); live bacterial vectors (e.g., Salmonella, Escherichia coli, bacillus Calmette-Guérin, Shigella, Lactobacillus) (Hone et al., 1996; Pouwels et al., 1998; Chatfield et al., 1993; Stover et al., 1991; Nugent et al., 1998); live viral vectors (e.g., Vaccinia, adenovirus, Herpes Simplex) (Gallichan et al., 1993, 1995; Moss et al., 1996; Nugent et al., 1998; Flexner et al., 1988; Morrow et al., 1999); microspheres (Gupta et al., 1998; Jones et al., 1996; Maloy et al., 1994; Moore et al., 1995; O'Hagan et al., 1994; Eldridge et al., 1989); nucleic acid vaccines (Fynan et al., 1993; Kuklin et al., 1997; Sasaki et al., 1998; Okada et al., 1997; Ishii et al., 1997); polymers (e.g., carboxymethylcellulose, chitosan) (Hamajima et al., 1998; Jabbal-Gill et al., 1998); polymer rings (Wyatt et al., 1998); proteosomes (Vancott et al., 1998; Lowell et al., 1988, 1996, 1997); sodium fluoride (Hashi et al., 1998); transgenic plants (Tacket et al., 1998; Mason et al., 1998; Haq et al., 1995); virosomes (Gluck et al., 1992; Mengiardi et al., 1995; Cryz et al., 1998); virus-like particles (Jiang et al., 1999; Leibl et al., 1998). Other delivery vehicles are known in the art and some additional examples are contemplated by the invention.

Combined with the teachings provided herein, by choosing among the various active compounds and weighing factors such as potency, relative bioavailability, subject body weight, severity of adverse side-effects and selected mode of administration, an effective prophylactic or therapeutic treatment regimen can be planned which does not cause substantial toxicity and yet is entirely effective to treat the particular subject. The effective amount for any particular application can vary depending on such factors as the disease or condition being treated, the particular agent being administered, the size of the subject, or the severity of the disease or condition. One of ordinary skill in the art can empirically determine the effective amount of a particular inhibitory oligonucleotide and/or small molecule antagonist of immunostimulatory CpG nucleic acids and/or other therapeutic agent without necessitating undue experimentation.

For any compound or combination of compounds described herein the therapeutically effective amount can be initially determined from animal models. A therapeutically effective dose can also be determined from human data for oligonucleotides which have been tested in humans and for compounds which are known to exhibit similar pharmacological activities. The applied dose can be adjusted based on the relative bioavailability and potency of the administered compound. Adjusting the dose to achieve maximal efficacy based on clinical or biological response and other methods as are well-known in the art is well within the capabilities of the ordinarily skilled artisan.

Formulations of the nucleic acid molecules and/or small molecule antagonists of immunostimulatory CpG nucleic acids of the invention are optionally administered in pharmaceutically acceptable solutions, which may routinely contain pharmaceutically acceptable concentrations of salt, buffering agents, preservatives, compatible carriers, and optionally other therapeutic ingredients.

Suitable liquid or solid pharmaceutical preparation forms are, for example, aqueous or saline solutions for inhalation, microencapsulated, encochleated, coated onto microscopic gold particles, contained in liposomes, nebulized, aerosols, pellets for implantation into the skin, or dried onto a sharp object to be scratched into the skin. The pharmaceutical compositions also include granules, powders, tablets, coated tablets, (micro)capsules, suppositories, syrups, emulsions, suspensions, creams, drops or preparations with protracted release of active compounds, in whose preparation excipients and additives and/or auxiliaries such as disintegrants, binders, coating agents, swelling agents, lubricants, flavorings, sweeteners or solubilizers are customarily used in pharmaceutical arts. The pharmaceutical compositions are suitable for use in a variety of drug delivery systems. For a brief review of methods for drug delivery, see Langer R (1990) Science 249:1527-33, which is incorporated herein by reference.

The inhibitory nucleic acid molecules and/or small molecule antagonists of immunostimulatory CpG nucleic acids and optionally other therapeutics and/or antigens may be administered per se (neat) or in the form of a pharmaceutically acceptable salt. When used in medicine, the salts should be pharmaceutically acceptable, but non-pharmaceutically acceptable salts may conveniently be used to prepare pharmaceutically acceptable salts thereof. Such salts include, but are not limited to, those prepared from the following acids: hydrochloric, hydrobromic, sulphuric, nitric, phosphoric, maleic, acetic, salicylic, p-toluene sulphonic, tartaric, citric, methane sulphonic, formic, malonic, succinic, naphthalene-2-sulphonic, and benzene sulphonic. Also, such salts can be prepared as alkaline metal or alkaline earth salts, such as sodium, potassium or calcium salts of the carboxylic acid group.

Suitable buffering agents include: acetic acid and a salt (1-2% w/v); citric acid and a salt (1-3% w/v); boric acid and a salt (0.5-2.5% w/v); and phosphoric acid and a salt (0.8-2% w/v). Suitable preservatives include benzalkonium chloride (0.003-0.03% w/v); chlorobutanol (0.3-0.9% w/v); parabens (0.01-0.25% w/v) and thimerosal (0.004-0.02% w/v).

For oral administration, the compounds (i.e., inhibitory oligonucleotides, small molecule antagonists of immunostimulatory CpG nucleic acids, antigens and other therapeutic agents, and combinations thereof) can be formulated readily by combining the active compound(s) with pharmaceutically acceptable carriers well known in the art. Such carriers enable the compounds of the invention to be formulated as tablets, pills, dragees, capsules, liquids, gels, syrups, slurries, suspensions and the like, for oral ingestion by a subject to be treated. Pharmaceutical preparations for oral use can be obtained as solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Optionally the oral formulations may also be formulated in saline or buffers for neutralizing internal acid conditions or may be administered without any carriers.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Microspheres formulated for oral administration may also be used. Such microspheres have been well defined in the art. All formulations for oral administration should be in dosages suitable for such administration.

For buccal administration, the compositions may take the form of tablets or lozenges formulated in conventional manner.

For administration by inhalation, the compounds for use according to the present invention may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g., gelatin for use in an inhaler or insufflator may be formulated containing a powder mix of the compound and a suitable powder base such as lactose or starch.

The compounds, when it is desirable to deliver them systemically, may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Alternatively, the active compounds may be in powder form for constitution with a suitable vehicle, e.g., sterile pyrogen-free water, before use.

The compounds may also be formulated in rectal or vaginal compositions such as suppositories or retention enemas, e.g., containing conventional suppository bases such as cocoa butter or other glycerides.

In addition to the formulations described previously, the compounds may also be formulated as a depot preparation. Such long acting formulations may be formulated with suitable polymeric or hydrophobic materials (for example as an emulsion in an acceptable oil) or ion exchange resins, or as sparingly soluble derivatives, for example, as a sparingly soluble salt.

The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

The term “pharmaceutically-acceptable carrier” means one or more compatible solid or liquid filler, diluents or encapsulating substances which are suitable for administration to a human or other vertebrate animal. The term “carrier” denotes an organic or inorganic ingredient, natural or synthetic, with which the active ingredient is combined to facilitate the application. The components of the pharmaceutical compositions also are capable of being commingled with the compounds of the present invention, and with each other, in a manner such that there is no interaction which would substantially impair the desired pharmaceutical efficiency.

The inhibitory oligonucleotides and small molecule antagonists of immunostimulatory CpG nucleic acids useful in the invention may be delivered in mixtures with adjuvant(s), other therapeutics, or antigen(s). A mixture may consist of several adjuvants in addition to the inhibitory oligonucleotide and small molecule antagonists of immunostimulatory CpG nucleic acids or several antigens or other therapeutics.

The particular mode selected will depend, of course, upon the particular active compounds selected, the particular condition being treated and the dosage required for therapeutic efficacy. The methods of this invention, generally speaking, may be practiced using any mode of administration that is medically acceptable, meaning any mode that inhibits effective levels of an immune response without causing clinically unacceptable adverse effects. Preferred modes of administration are discussed above.

The compositions may conveniently be presented in unit dosage form and may be prepared by any of the methods well known in the art of pharmacy. All such methods include the step of bringing the compounds into association with a carrier which constitutes one or more accessory ingredients. In general, the compositions are prepared by uniformly and intimately bringing the compounds into association with a liquid carrier, a finely divided solid carrier, or both, and then, if necessary, shaping the product. Liquid dose units are vials or ampoules. Solid dose units are tablets, capsules and suppositories. For treatment of a patient, depending on activity of the compound, manner of administration, purpose of the immunization (i.e., prophylactic or therapeutic), nature and severity of the disorder, age and body weight of the patient, different doses may be necessary. The administration of a given dose can be carried out both by single administration in the form of an individual dose unit or else several smaller dose units. Multiple administration of doses at specific intervals of weeks or months apart is usual for boosting the antigen-specific responses.

Other delivery systems can include time-release, delayed release or sustained release delivery systems. Such systems can avoid repeated administrations of the compounds, increasing convenience to the subject and the physician. Many types of release delivery systems are available and known to those of ordinary skill in the art. They include polymer base systems such as poly(lactide-glycolide), copolyoxalates, polycaprolactones, polyesteramides, polyorthoesters, polyhydroxybutyric acid, and polyanhydrides. Microcapsules of the foregoing polymers containing drugs are described in, for example, U.S. Pat. No. 5,075,109. Delivery systems also include non-polymer systems that are: lipids including sterols such as cholesterol, cholesterol esters and fatty acids or neutral fats such as mono-di-and tri-glycerides; hydrogel release systems; sylastic systems; peptide based systems; wax coatings; compressed tablets using conventional binders and excipients; partially fused implants; and the like. Specific examples include, but are not limited to: (a) erosional systems in which an agent of the invention is contained in a form within a matrix such as those described in U.S. Pat. Nos. 4,452,775, 4,675,189, and 5,736,152, and (b) diffusional systems in which an active component permeates at a controlled rate from a polymer such as described in U.S. Pat. Nos. 3,854,480, 5,133,974 and 5,407,686. In addition, pump-based hardware delivery systems can be used, some of which are adapted for implantation.

Each of the foregoing lists is illustrative, and is not intended to be limiting.

### EXAMPLES

### Example 1

### Reconstitution of TLR₉ Signaling in 293 Fibroblasts

Methods for cloning murine and human TLR₉ have been described in pending U.S. patent application Ser. No. 09/954,987 and corresponding published PCT application PCT/US01/29229 (WO 02/22809), both filed Sep. 17, 2001, the contents of which are incorporated by reference. Human TLR₉ cDNA and murine TLR₉ cDNA in pT-Adv vector (from Clonetech) were individually cloned into the expression vector pcDNA3.1 (−) from Invitrogen using the EcoRI site. Utilizing a “gain of function” assay it was possible to reconstitute human TLR₉ (hTLR₉) and murine TLR₉ (mTLR₉) signaling in CpG-DNA non-responsive human 293 fibroblasts (ATCC, CRL-1573). The expression vectors mentioned above were transfected into 293 fibroblast cells using the calcium phosphate method.

| cDNA Sequence for Human TLR9 (GenBank Accession No. AF245704) | |
|---|---|
| SEQ ID NO:21 | |
| aggctggtat aaaaatctta cttcctctat tctctgagcc gctgctgccc ctgtgggaag | 60 |
| ggacctcgag tgtgaagcat ccttccctgt agctgctgtc cagtctgccc gccagaccct | 120 |
| ctggagaagc ccctgccccc cagcatgggt ttctgccgca gcgccctgca cccgctgtct | 180 |
| ctcctggtgc aggccatcat getggccatg accctggccc tgggtacctt gcctgccttc | 240 |
| ctaccctgtg agctccagcc ccacggcctg gtgaactgca actggctgtt cctgaagtct | 300 |
| gtgccccact tctccatggc agcaccccgt ggcaatgtca ccagcctttc cttgtcctcc | 360 |
| aaccgcatcc accacctcca tgattctgac tttgcccacc tgcccagcct gcggcatctc | 420 |
| aacctcaagt ggaactgccc gccggttggc ctcagcccca tgcacttccc ctgccacatg | 480 |
| accatcgagc ccagcacctt cttggctgtg cccaccctgg aagagctaaa cctgagctac | 540 |
| aacaacatca tgactgtgcc tgcgctgccc aaatccctca tatccctgtc cctcagccat | 600 |
| accaacatcc tgatgctaga ctctgccagc ctcgccggcc tgcatgccct gcgcttccta | 660 |
| ttcatggacg gcaactgtta ttacaagaac ccctgcaggc aggcactgga ggtggccccg | 720 |
| ggtgccctcc ttggcctggg caacctcacc cacctgtcac tcaagtacaa caacctcact | 780 |
| gtggtgcccc gcaacctgcc ttccagcctg gagtatctgc tgttgtccta caaccgcatc | 840 |
| gtcaaactgg cgcctgagga cctggccaat ctgaccgccc tgcgtgtgct cgatgtgggc | 900 |
| ggaaattgcc gccgctgcga ccacgctccc aacccctgca tggagtgccc tcgtcacttc | 960 |
| ccccagctac atcccgatac cttcagccac ctgagccgtc ttgaaggcct ggtgttgaag | 1020 |
| gacagttctc tctcctggct gaatgccagt tggttccgtg ggctgggaaa cctccgagtg | 1080 |
| ctggacctga gtgagaactt cctctacaaa tgcatcacta aaaccaaggc cttccagggc | 1140 |
| ctaacacagc tgcgcaagct taacctgtcc ttcaattacc aaaagagggt gtcctttgcc | 1200 |
| cacctgtctc tggccccttc cttcgggagc ctggtcgccc tgaaggagct ggacatgcac | 1260 |
| ggcatcttct tccgctcact cgatgagacc acgctccggc cactggcccg cctgcccatg | 1320 |
| ctccagactc tgcgtctgca gatgaacttc atcaaccagg cccagctcgg catcttcagg | 1380 |
| gccttccctg gcctgcgcta cgtggacctg tcggacaacc gcatcagcgg agcttcggag | 1440 |
| ctgacagcca ccatggggga ggcagatgga ggggagaagg tctggctgca gcctggggac | 1500 |
| cttgctccgg ccccagtgga cactcccagc tctgaagact tcaggcccaa ctgcagcacc | 1560 |
| ctcaacttca ccttggatct gtcacggaac aacctggtga ccgtgcagcc ggagatgttt | 1620 |
| gcccagctct cgcacctgca gtgcctgcgc ctgagccaca actgcatctc gcaggcagtc | 1680 |
| aatggctccc agttcctgcc gctgaccggt ctgcaggtgc tagacctgtc ccgcaataag | 1740 |
| ctggacctct accacgagca ctcattcacg gagctaccgc gactggaggc cctggacctc | 1800 |
| agctacaaca gccagccctt tggcatgcag ggcgtgggcc acaacttcag cttcgtggct | 1860 |
| cacctgcgca ccctgcgcca cctcagcctg gcccacaaca acatccacag ccaagtgtcc | 1920 |
| cagcagctct gcagtacgtc gctgcgggcc ctggacttca gcggcaatgc actgggccat | 1980 |
| atgtgggccg agggagacct ctatctgcac ttcttccaag gcctgagcgg tttgatctgg | 2040 |
| ctggacttgt cccagaaccg cctgcacacc ctcctgcccc aaaccctgcg caacctcccc | 2100 |
| aagagcctac aggtgctgcg tctccgtgac aattacctgg ccttctttaa gtggtggagc | 2160 |
| ctccacttcc tgcccaaact ggaagtcctc gacctggcag gaaaccggct gaaggccctg | 2220 |
| accaatggca gcctgcctge tggcacccgg ctccggaggc tggatgtcag ctgcaacagc | 2280 |
| atcagcttcg tggcccccgg cttcttttcc aaggccaagg agctgcgaga gctcaacctt | 2340 |
| agcgccaacg ccctcaagac agtggaccac tcctggtttg ggcccctggc gagtgccctg | 2400 |
| caaatactag atgtaagcgc caaccctctg cactgcgcct gtggggcggc ctttatggac | 2460 |
| ttcctgctgg aggtgcaggc tgccgtgccc ggtctgccca gccgggtgaa gtgtggcagt | 2520 |
| ccgggccagc tccagggcct cagcatcttt gcacaggacc tgcgcctctg cctggatgag | 2580 |
| gccctctcct gggactgttt cgccctctcg ctgctggctg tggctctggg cctgggtgtg | 2640 |
| cccatgctgc atcacctctg tggctgggac ctctggtact gcttccacct gtgcctggcc | 2700 |
| tggcttccct ggcgggggcg gcaaagtggg cgagatgagg atgccctgcc ctacgatgcc | 2760 |
| ttcgtggtct tcgacaaaac gcagagcgca gtggcagact gggtgtacaa cgagcttcgg | 2820 |
| gggcagctgg aggagtgccg tgggcgctgg gcactccgcc tgtgcctgga ggaacgcgac | 2880 |
| tggctgcctg gcaaaaccct ctttgagaac ctgtgggcct cggtctatgg cagccgcaag | 2940 |
| acgctgtttg tgctggccca cacggaccgg gtcagtggtc tcttgcgcgc cagcttcctg | 3000 |
| ctggcccagc agcgcctgct ggaggaccgc aaggacgtcg tggtgctggt gatcctgagc | 3060 |
| cctgacggcc gccgctcccg ctacgtgcgg ctgcgccagc gcctctgccg ccagagtgtc | 3120 |
| ctcctctggc cccaccagcc cagtggtcag cgcagcttct gggcccagct gggcatggcc | 3180 |
| ctgaccaggg acaaccacca cttctataac cggaacttct gccagggacc cacggccgaa | 3240 |
| tagccgtgag ccggaatcct gcacggtgcc acctccacac tcacctcacc tctgcctgcc | 3300 |
| tggtctgacc ctcccctgct cgcctccctc accccacacc tgacacagag ca | 3352 |
| Amino Acid Sequence for Human TLR9 (GenBank Accession No. AAF78037) | |
| SEQ ID NO:22 | |
| MGFCRSALHP LSLLVQAIML AMTLALGTLP AFLPCELQPH GLVNCNWLFL KSVPHFSMAA | 60 |
| PRGNVTSLSL SSNRIHHLHD SDFAHLPSLR HLNLKWNCPP VGLSPMHFPC HMTIEPSTFL | 120 |
| AVPTLEELNL SYNNIMTVPA LPKSLISLSL SHTNILMLDS ASLAGLHALR FLFMDGNCYY | 180 |
| KNPCRQALEV APGALLGLGN LTHLSLKYNN LTVVPRNLPS SLEYLLLSYN RIVKLAPEDL | 240 |
| ANLTALRVLD VGGNCRRCDH APNPCMECPR HFPQLHPDTF SHLSRLEGLV LKDSSLSWLN | 300 |
| ASWFRGLGNL RVLDLSENFL YKCITKTKAF QGLTQLRKLN LSFNYQKRVS FAHLSLAPSF | 360 |
| GSLVALKELD MHGIFFRSLD ETTLRPLARL PMLQTLRLQM NFINQAQLGI FRAFPGLRYV | 420 |
| DLSDNRISGA SELTATMGEA DGCEKVWLQP GDLAPAPVDT PSSEDFRPNC STLNFTLDLS | 480 |
| RNNLVTVQPE MFAQLSHLQC LRLSHNCISQ AVNGSQFLPL TGLQVLDLSR NKLDLYHEHS | 540 |
| FTELPRLEAL DLSYNSQPFG MQGVGHNFSF VAHLRTLRHL SLAHNNIHSQ VSQQLCSTSL | 600 |
| RALDFSGNAL GHMWAEGDLY LHFFQGLSGL IWLDLSQNRL HTLLPQTLRN LPKSLQVLRL | 660 |
| RDNYLAFFKW WSLHFLPKLE VLDLACNRLK ALTNGSLPAG TRLRRLDVSC NSISFVAPGF | 720 |
| FSKAKELREL NLSANALKTV DHSWFGPLAS ALQILDVSAN PLHCACGAAF MDFLLEVQAA | 780 |
| VPGLPSRVKC GSPGQLQGLS IFAQDLRLCL DEALSWDCFA LSLLAVALGL GVPMLHHLCG | 840 |
| WDLWYCFHLC LAWLPWRGRQ SGRDEDALPY DAFVVFDKTQ SAVADWVYNE LRGQLEECRG | 900 |
| RWALRLCLEE RDWLPGKTLF ENLWASVYGS RKTLFVLAHT DRVSGLLRAS FLLAQQRLLE | 960 |
| DRKDVVVLVI LSPDGRRSRY VRLRQRLCRQ SVLLWPHQPS GQRSFWAQLG MALTRDNHHF | 1020 |
| YNRNFCQGPT AE | 1032 |
| cDNA Sequence for Murine TLR9 (GenBank Accession No. AF348140) | |
| SEQ ID NO:23 | |
| tgtcagaggg agcctcggga gaatcctcca tctcccaaca tggttctccg tcgaaggact | 60 |
| ctgcacccct tgtccctcct ggtacaggct gcagtgctgg ctgagactct ggccctgggt | 120 |
| accctgcctg ccttcctacc ctgtgagctg aagcctcatg gcctggtgga ctgcaattgg | 180 |
| ctgttcctga agtctgtacc ccgtttctct gcggcagcat dctgctccaa catcacccgc | 240 |
| ctctccttga tctccaaccg tatccaccac ctgcacaact ccgacttcgt ccacctgtcc | 300 |
| aacctgcggc agctgaacct caagtggaac tgtccaccca ctggccttag ccccctgcac | 360 |
| ttctcttgcc acatgaccat tgagcccaga accttcctgg ctatgcgtac actggaggag | 420 |
| ctgaacctga gctataatgg tatcaccact gtgccccgac tgcccagctc cctggtgaat | 480 |
| ctgagcctga gccacaccaa catcctggtt ctagatgcta acagcctcgc cggcctatac | 540 |
| agcctgcgcg ttctcttcat ggacgggaac tgctactaca agaacccctg cacaggagcg | 600 |
| gtgaaggtga ccccaggcgc cctcctgggc ctgagcaatc tcacccatct gtctctgaag | 660 |
| tataacaacc tcacaaaggt gccccgccaa ctgcccccca gcctggagta cctcctggtg | 720 |
| tcctataacc tcattgtcaa gctggggcct gaagacctgg ccaatctgac ctcccttcga | 780 |
| gtacttgatg tgggtgggaa ttgccgtcgc tgcgaccatg cccccaatcc ctgtatagaa | 840 |
| tgtggccaaa agtccctcca cctgcaccct gagaccttcc atcacctgag ccatctggaa | 900 |
| ggcctggtgc tgaaggacag ctctctccat acactgaact cttcctggtt ccaaggtctg | 960 |
| gtcaacctct cggtgctgga cctaagcgag aactttctct atgaaagcat caaccacacc | 1020 |
| aatgcctttc agaacctaac ccgcctgcgc aagctcaacc tgtccttcaa ttaccgcaag | 1080 |
| aaggtatcct ttgcccgcct ccacctggca agttccttca agaacctggt gtcactgcag | 1140 |
| gagctgaaca tgaacggcat cttcttccgc tcgctcaaca agtacacgct cagatggctg | 1200 |
| gccgatctgc ccaaactcca cactctgcat cttcaaatga acttcatcaa ccaggcacag | 1260 |
| ctcagcatct ttggtacctt ccgagccctt cgctttgtgg acttgtcaga caatcgcatc | 1320 |
| agtgggcctt caacgctgtc agaagccacc cctgaagagg cagatgatgc agagcaggag | 1380 |
| gagctgttgt ctgcggatcc tcacccagct ccactgagca cccctgcttc taagaacttc | 1440 |
| atggacaggt gtaagaactt caagttcacc atggacctgt ctcggaacaa cctggtgact | 1500 |
| atcaagccag agatgtttgt caatctctca cgcctccagt gtcttagcct gagccacaac | 1560 |
| tccattgcac aggctgtcaa tggctcteag ttcctgccgc tgactaatct gcaggtgctg | 1620 |
| gacctgtccc ataacaaact ggacttgtac cactggaaat cgttcagtga gctaccacag | 1680 |
| ttgcaggccc tggacctgag ctacaacagc cagcccttta gcatgaaggg tataggccac | 1740 |
| aatttcagtt ttgtggccca tctgtccatg ctacacagcc ttagcctggc acacaatgac | 1800 |
| attcataccc gtgtgtcctc acatctcaac agcaactcag tgaggtttct tgacttcagg | 1860 |
| ggcaacggta tgggccgcat gtgggatgag gggggccttt atctccattt cttccaaggc | 1920 |
| ctgagtggcc tgctgaagct ggacctgtct caaaataacc tgcatatcct ccggccccag | 1980 |
| aaccttgaca acctccccaa gagcctgaag ctgctgagcc tccgagacaa ctacctatct | 2040 |
| ttctttaact ggaccagtct gtccttcctg cccaacctgg aagtcctaga cctggcaggc | 2100 |
| aaccagctaa aggccctgac caatggcacc ctgcctaatg gcaccctcct ccagaaactg | 2160 |
| gatgtcagca gcaacagtat cgtctctgtg gtcccagcct tcttcgctct ggcggtcgag | 2220 |
| ctgaaagagg tcaacctcag ccacaacatt ctcaagacgg tggatcgctc ctggtttggg | 2280 |
| cccattgtga tgaacctgac agttctagac gtgagaagca accctctgca ctgtgcctgt | 2340 |
| ggggcagcct tcgtagactt actgttggag gtgcagacca aggtgcctgg cctggctaat | 2400 |
| ggtgtgaagt gtggcagccc cggccagctg cagggccgta gcatcttcgc acaggacctg | 2460 |
| cggctgtgcc tggatgaggt cctctcttgg gactgctttg gcctttcact cttggctgtg | 2520 |
| gccgtgggca tggtggtgcc tatactgcac catctctgcg gctgggacgt ctggtactgt | 2580 |
| tttcatctgt gcctggcatg gctacctttg ctggcccgca gccgacgcag cgcccaagct | 2640 |
| ctcccctatg atgccttcgt ggtgttcgat aaggcacaga gcgcagttgc ggactgggtg | 2700 |
| tataacgagc tgcgggtgcg gctggaggag cggcgcggtc gccgagccct acgcttgtgt | 2760 |
| ctggaggacc gagattggct gcctggccag acgctcttcg agaacctctg ggcttccatc | 2820 |
| tatgggagcc gcaagactct atttgtgctg gcccacacgg accgcgtcag tggcctcctg | 2880 |
| cgcaccagct tcctgctggc tcagcagcgc ctgttggaag accgcaagga cgtggtggtg | 2940 |
| ttggtgatcc tgcgtccgga tgcccaccgc tcccgctatg tgcgactgcg ccagcgtctc | 3000 |
| tgccgccaga gtgtgctctt ctggccccag cagcccaacg ggcagggggg cttctgggcc | 3060 |
| cagctgagta cagccctgac tagggacaac cgccacttct ataaccagaa cttctgccgg | 3120 |
| ggacctacag cagaatagct cagagcaaca gctggaaaca gctgcatctt catgcctggt | 3180 |
| tcccgagttg ctctgcctgc | 3200 |
| Amino Acid Sequence for Murine TLR9 (GenBank Accession No. AAK29625) | |
| SEQ ID NO:24 | |
| MVLRRRTLHP LSLLVQAAVL AETLALGTLP AFLPCELKPH GLVDCNWLFL KSVPRFSAAA | 60 |
| SCSNITRLSL ISNRIHHLHN SDFVHLSNLR QLNLKWNCPP TGLSPLHFSC HMTIEPRTFL | 120 |
| ADRTLEELNL SYNGITTVPR LPSSLVMLSL SHTNILVLDA NSLAGLYSLR VLFMDGNCYY | 180 |
| KNPCTGAVKV TPGALLGLSN LTHLSLKYNN LTKVPRQLPP SLEYLLVSYN LIVKLGPEDL | 240 |
| ANLTSLRVLD VGGNCRRCDH APNPCIECGQ KSLHLHPETF HHLSHLEGLV LKDSSLHTLN | 300 |
| SSWFQGLVNL SVLDLSENFL YESINHTNAF QNLTRLRKLN LSFNYRKKVS FARLHLASSF | 360 |
| KNLVSLQELN MNGIFFRSLN KYTLRWLADL PKLHTLHLQM NFINQAQLSI FGTFRALRFV | 420 |
| DLSDNRISGP STLSEATPEE ADDAEQEELL SADPHPAPLS TPASKNFMDR CKNFKFTMDL | 480 |
| SRNNLVTIKP EMFVNLSRLQ CLSLSHNSIA QAVNGSQFLP LTNLQVLDLS HNKLDLYHWK | 540 |
| SFSELPQLQA LDLSYNSQPF SMKGIGHNFS FVAHLSMLHS LSLAHNDIHT RVSSHLNSNS | 600 |
| VRFLDFSGNG MGRMWDEGGL YLHFFQGLSG LLKLDLSQNN LHILRPQNLD NLPKSLKLLS | 660 |
| LRDNYLSFFN WTSLSFLPNL EVLDLAGNQL KALTNGTLPN GTLLQKLDVS SNSIVSVVPA | 720 |
| FEALAVELKE VNLSHNILKT VDRSWFGPIV MNLTVLDVRS NPLHCACGAA FVDLLLEVQT | 780 |
| KVPGLANGVK CGSPGQLQGR SIFAQDLRLC LDEVLSWDCF GLSLLAVAVG MVVPILHHLC | 840 |
| GWDVWYCFHL CLAWLPLLAR SRRSAQALPY DAFVVFDKAQ SAVADWVYNE LRVRLEERRG | 900 |
| RRALRLCLED RDWLPGQTLF ENLWASIYGS RKTLFVLAHT DRVSGLLRTS FLLAQQRLLE | 960 |
| DRKDVVVLVI LRPDAHRSRY VRLRQRLCRQ SVLFWPQQPN GQGGFWAQLS TALTRDNRHF | 1020 |
| YNQNFCRGPT AE | 1032 |

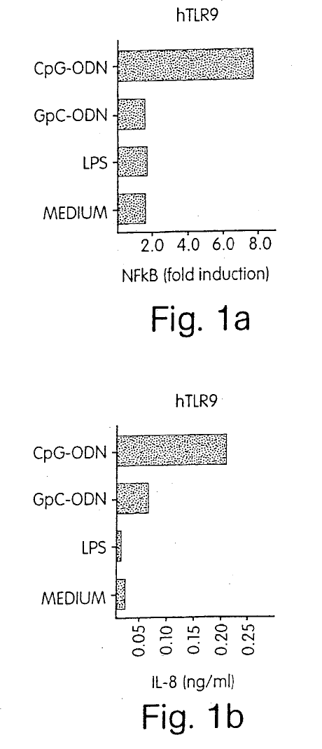

Since NF-κB activation is central to the IL-1/TLR signal transduction pathway (Medzhitov R et al. (1998) Mol Cell 2:253-8; Muzio M et al. (1998) J Exp Med 187:2097-101), cells were transfected with hTLR₉ or co-transfected with hTLR₉ and an NF-κB-driven luciferase reporter construct. Human 293 fibroblast cells were transiently transfected with (FIG. 1A) hTLR₉ and a 6×NF-κB-luciferase reporter plasmid (NF-κB-luc, kindly provided by Patrick Baeuerle, Munich, Germany) or (FIG. 1B) with hTLR₉ alone. After stimulus with CpG-ODN (2006, 2 μM, TCGTCGTTTTGTCGTTTTGTCGTT, SEQ ID NO:25), GpC-ODN (2006-GC, 2 μM, TGCTGCTTTTGTGCTTTTGTGCTT, SEQ ID NO:26), LPS (100 ng/mL) or media, NF-κB activation by luciferase readout (8 h, FIG. 1A) or IL-8 production by ELISA (48 h, FIG. 1B) were monitored. Results are representative of three independent experiments. FIG. 1 shows that cells expressing hTLR₉ responded to CpG-DNA but not to LPS.

FIG. 2 demonstrates the same principle for the transfection of mTLR_{9.} Human 293 fibroblast cells were transiently transfected with mTLR₉ and the NF-κB-luc construct (FIG. 2). Similar data was obtained for IL-8 production (not shown). Thus expression of TLR₉ (human or mouse) in 293 cells results in a gain of function for CpG-DNA stimulation similar to hTLR₄ reconstitution of LPS responses.

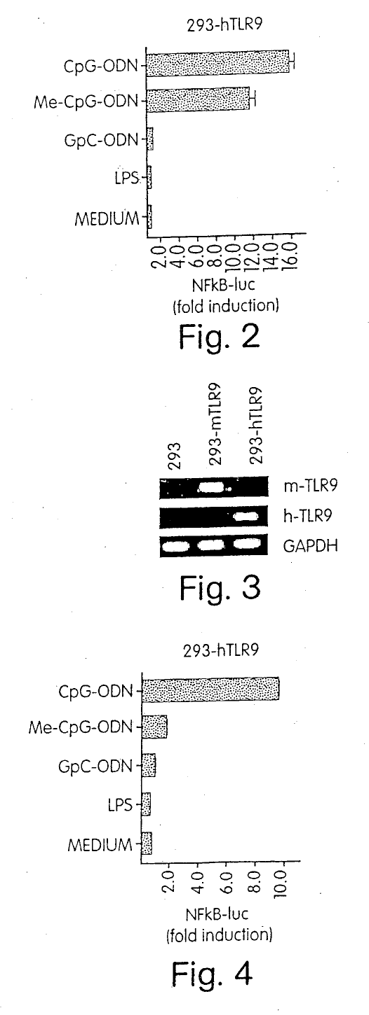

To generate stable clones expressing human TLR₉, murine TLR₉, or either TLR₉ with the NF-κB-luc reporter plasmid, 293 cells were transfected in 10 cm plates (2×10⁶ cells/plate) with 16 μg of DNA and selected with 0.7 mg/mL G418 (PAA Laboratories GmbH, Cölbe, Germany). Clones were tested for TLR₉ expression by RT-PCR, for example as shown in FIG. 3. The clones were also screened for IL-8 production or NF-κB-luciferase activity after stimulation with ODN. Four different types of clones were generated.

| | |
|---|---|
| 293-hTLR9-luc: | expressing human TLR9 and 6x NF-κB-luciferase |
| | reporter |
| 293-mTLR9-luc: | expressing murine TLR9 and 6x NF-κB-luciferase |
| | reporter |
| 293-hTLR9: | expressing human TLR9 |
| 293-mTLR9: | expressing murine TLR9 |

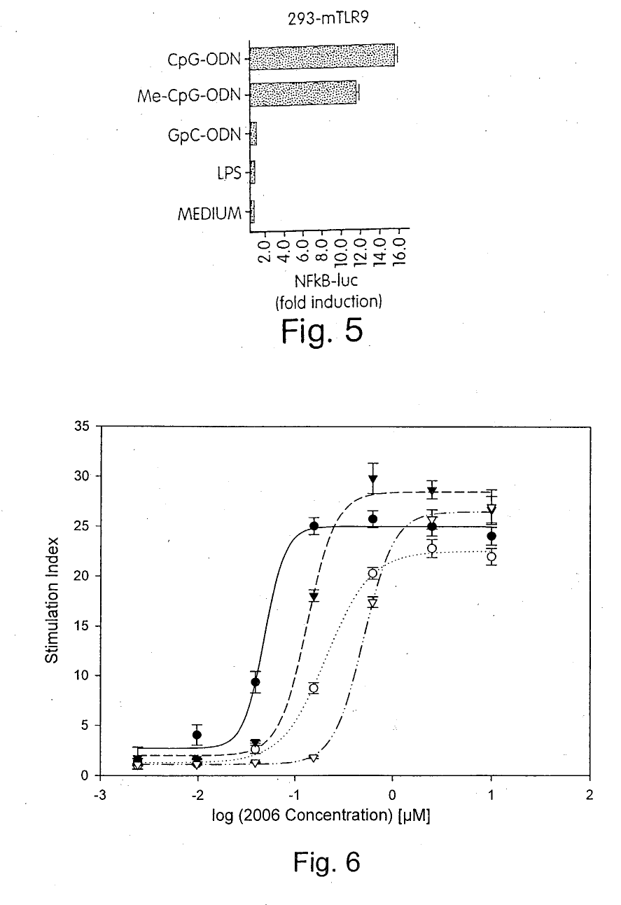

FIG. 4 demonstrates the responsiveness of a stable 293-hTLR₉-luc clone after stimulation with CpG-ODN (2006, 2 μM), GpC-ODN (2006-GC, 2 μM), Me-CpG-ODN (2006 methylated, 2 μM; TZGTZGTTTTGTZGTTTTGTZGTT, Z=5-methylcytidine, SEQ ID NO:27), LPS (100 ng/mL) or media, as measured by monitoring NF-κB activation. Similar results were obtained utilizing IL-8 production with the stable clone 293-hTLR_{9. 293}-mTLR₉-luc were also stimulated with CpG-ODN (1668, 2 μM; TCCATGACGTTCCTGATGCT, SEQ ID NO:28), GpC-ODN (1668-GC, 2 μM; TCCATGAGCTTCCTGATGCT, SEQ ID NO:29), Me-CpG-ODN (1668 methylated, 2 μM; TCCATGAZGTTCCTGATGCT, Z=5-methylcytidine, SEQ ID NO:30), LPS (100 ng/mL) or media, as measured by monitoring NF-κB activation (FIG. 5). Similar results were obtained utilizing IL-8 production with the stable clone 293-mTLR_{9.} Results are representative of at least two independent experiments. These results demonstrate that CpG-DNA non-responsive cell lines can be stably genetically complemented with TLR₉ to become responsive to CpG-DNA in a motif-specific manner.

### Example 2

### Synergistic Inhibition of CpG-Mediated TLR₉ Activation by a Combination of Inhibitory ODN and Inhibitor of Endosomal Acidification/Maturation

Stably transfected 293-hTLR₉-luc cells from Example 1 were incubated with various amounts of CpG ODN 2006 in the presence of absence of a constant amount of inhibitory ODN 2088 (SEQ ID NO:7) and/or chloroquine. NF-κB activation was measured by determining luciferase activity of cells 16 h later. Results are given as fold induction above medium background.

Table 1 shows the concentration at which activation by ODN 2006 is 50 percent maximal (EC₅₀) in the presence of buffer alone (TE), ODN 2088 alone, chloroquine alone, or ODN 2088 plus chloroquine, where chloroquine is added at three different concentrations (ca. 1000 nM, 250 nM, and 125 nM). It is evident from this table that while either agent alone effectively increased the EC₅₀ of CpG ODN 2006, the combination of inhibitory ODN 2008 plus chloroquine potently further increased the EC₅₀ of ODN 2006. This synergistic effect is shown in Table 2, which presents fold increase of EC₅₀ compared to ODN 2006 plus TE. As shown in Table 2, the observed fold increase in EC₅₀ exceeds the expected fold increase in EC₅₀ if the effect of each agent were merely additive.

TABLE 1
EC₅₀ (nM) of CpG ODN 2006

| ODN 2088 added at | 0.05 μM | 0.05 μM | 0.05 μM |
|---|---|---|---|
| Chloroquine added at | 0.3125 μg/mL | 0.078 μg/mL | 0.039 μg/mL |
| +TE | 50 | 50 | 30 |
| +2088 | 400 | 210 | 440 |
| +Chloroquine | 180 | 130 | 70 |
| +2088 + Chloroquine | 820 | 520 | 720 |

TABLE 2
Fold increase of EC₅₀ compared to ODN 2006 + TE

| | | | |
|---|---|---|---|
| +2088 | 8.0 | 4.2 | 14.7 |
| +Chloroquine | 3.6 | 2.6 | 2.3 |
| +2088 + Chloroquine | 16.4 | 10.4 | 24.0 |
| Expected if Additive | 11.6 | 6.8 | 17.0 |

FIG. 6 depicts the concentration-dependent stimulation index for ODN 2006 in the presence of TE alone, 0.05 μM ODN 2088 alone, 0.078 μg/mL chloroquine alone, and the combination of ODN 2088 and chloroquine at these same concentrations.

All of the references, patents and patent publications identified or cited herein are incorporated in their entirety by reference.

Although this invention has been described with respect to specific embodiments, the details of these embodiments are not to be construed as limitations. Various equivalents, changes and modifications can be made without departing from the spirit and scope of this invention, and it is understood that such equivalent embodiments are part of this invention.

I claim:

1. A method of inhibiting immune activation, comprising:
contacting a TLR₉-expressing cell with an inhibitory nucleic acid and a small molecule antagonist of immunostimulatory CpG nucleic acids, in an effective amount to inhibit activation of the TLR₉-expressing cell by a nucleic acid-containing immune complex.

2. The method of claim 1, wherein the TLR₉-expressing cell is chosen from a B cell, a plasmacytoid dendritic cell (pDC), an endothelial cell, and a macrophage.

3. The method of claim 1, wherein the TLR₉-expressing cell is a B cell.

4. The method of claim 1, wherein the TLR₉-expressing cell is a human cell.

5. A method of treating an autoimmune disease, comprising:
administering to a subject having or at risk of developing an autoimmune disease an inhibitory nucleic acid and a small molecule antagonist of immunostimulatory CpG nucleic acids, in an effective amount to treat or prevent the autoimmune disease.

6. The method of claim 5, wherein the autoimmune disease is chosen from rheumatoid arthritis (RA), systemic lupus erythematosus (SLE), inflammatory bowel disease (IBD), multiple sclerosis (MS), glomerulonephritis, type 1 diabetes mellitus, Sjögren's syndrome, viral infections associated with hepatitis B virus (HBV) and hepatitis C virus (HCV), graft-versus-host disease (GvHD), paraneoplastic autoimmune syndrome associated with small cell lung cancer, and paraneoplastic autoimmune syndrome associated with breast cancer.

7. The method of any one of claims 1-6, wherein the inhibitory nucleic acid comprises a poly G motif.

8. The method of claim 7, wherein the poly G motif comprises a sequence chosen from GGGG, N₁GGGN₂GGGN₃ (SEQ ID NO:20), wherein N₁, N₂, and N₃ are each independently any nucleic acid sequence comprising 0-20 nucleotides, a sequence of 5 nucleotides in which at least 4 nucleotides are G, a sequence of 7 nucleotides in which at least 5 nucleotides are G, and a sequence of 8 nucleotides in which at least 6 nucleotides are G.

9. The method of any one of claims 1-6, wherein the inhibitory nucleic acid comprises a sequence chosen from

| | |
|---|---|
| GTGCCGGGGTCTCCGGGC, | (SEQ ID NO:1) |
| GCTGTGGGGCGGCTCCTG, | (SEQ ID NO:2) |
| GGGGTCAACGTTGAGGGGGG, | (SEQ ID NO:3) |
| GGGGAGGGT, | (SEQ ID NO:4) |
| GGGGAGGGG, | (SEQ ID NO:5) |
| CACGTTGAGGGGCAT, | (SEQ ID NO:6) |
| TCCTGGCGGGGAAGT, | (SEQ ID NO:7) |
| TCCTGGAGGGGAAGT, | (SEQ ID NO:8) |
| GGCTCCGGGGAGGGAATTTTTGTCTAT, | (SEQ ID NO:9) |
| TCCTGCCGGGGAAGT, | (SEQ ID NO:10) |
| TCCTGCAGGGGAAGT, | (SEQ ID NO:11) |
| TCCTGAAGGGGAAGT, | (SEQ ID NO:12) |
| TCCTGGCGGGCAAGT, | (SEQ ID NO:13) |
| TCCTGGCGGGTAAGT, | (SEQ ID NO:14) |
| TCCTGGCGGGAAAGT, | (SEQ ID NO:15) |
| TCCGGGCGGGGAAGT, | (SEQ ID NO:16) |
| TCGGGGCGGGGAAGT, | (SEQ ID NO:17) |
| TCCCGGCGGGGAAGT, and | (SEQ ID NO:18) |
| GGGGGACGTTGGGGG. | (SEQ ID NO:19) |

10. The method of any one of claims 1-6, wherein the inhibitory nucleic acid comprises a stabilized backbone.

11. The method of claim 10, wherein the stabilized backbone is a phosphorothioate backbone.

12. The method of any one of claims 1-6, wherein the small molecule antagonist of immunostimulatory CpG nucleic acids is chosen from quinacrine, chloroquine, hydroxychloroquine, substituted 4-quinolinamines, 2-phenylquinolin-4-amines, 4-aminoquinolines, and 9-aminoacridines.

13. The method of any one of claims 1-6, wherein the small molecule antagonist of immunostimulatory CpG nucleic acids is chosen from compounds having structural Formula 1: 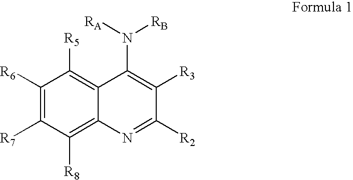
wherein R_{A} is a hydrogen atom, a lower alkyl group, or linked to R_{B} by a substituted or unsubstituted alkyl chain;
R_{B} is a hydrogen atom, an alicyclic group, an alkyl secondary, tertiary or quaternary amine, or an alkenyl secondary, tertiary or quaternary amine;
R₂ is a hydrogen atom, a lower alkyl group, an aryl group, a heteroaromatic group, or a lower alkenyl group substituted with an aryl group;
R₃ is a hydrogen atom, a lower alkyl group, or an aromatic group;
R₅ is a hydrogen atom, a lower alkyl group, or a halogen atom;
R₆ is a hydrogen atom, a lower alkyl group, a lower alkoxy group, an aryloxy group, an aryl group, an amino group, or a thioether group;
R₇ is a hydrogen atom, a lower alkyl group, a lower alkoxy group, an aryloxy group, a haloalkyl group, or a halogen atom; and
R₈ is a hydrogen group, or a lower alkoxy group, and
pharmaceutically acceptable salts thereof, with the proviso that if R₇ is a halogen, then at least one of R₂, R₃, R₅, R₆ or R₈ is non-hydrogen and R_{B} is not 4-[N,N-dialkyl-n-pentylamine] or 4-[N-alkyl-N-hydroxyalkyl-n-pentylamine].

14. The method of any one of claims 1-6, wherein the small molecule antagonist of immunostimulatory CpG nucleic acids is chosen from compounds having structural Formula 2: 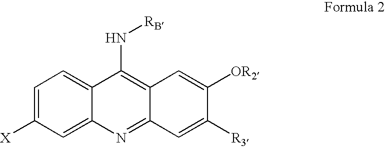
wherein R_{B′} is a hydrogen atom or an alkyl secondary, tertiary, or quaternary amino group;
R_{2′} is a lower alkyl group;
R_{3′} is a hydrogen atom or a lower alkoxy group;
X is a halogen atom; and
pharmaceutically acceptable salts thereof.

15. The method of any one of claims 1-6, wherein the small molecule antagonist of immunostimulatory CpG nucleic acids is a compound of Formula 3: 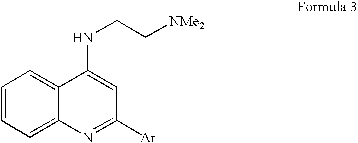
wherein Ar is selected from 2-naphthyl, 3-phenanthryl, 4-MePh, and trans-CH═CHPh.

16. The method of any one of claims 1-6, wherein the small molecule antagonist of immunostimulatory CpG nucleic acids is a compound of Formula 4: 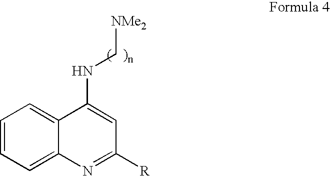
wherein n is an integer between 3 and 6, inclusive, and R is selected from p-tolyl or 2-naphthyl when n is 3, 2-naphthyl when n is 4, and 2-naphthyl when n is 6.

17. The method of any one of claims 1-6, wherein the small molecule antagonist of immunostimulatory CpG nucleic acids is a compound of Formula 5: 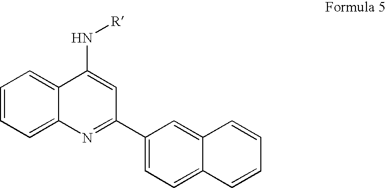
wherein R′ is (CH₂)₃N(CH₂CH₂)₂N(CH₂)₃NHC(O)(CH₂)₃OH.

18. The method of any one of claims 1-6, wherein the small molecule antagonist of immunostimulatory CpG nucleic acids is a compound of Formula 6: 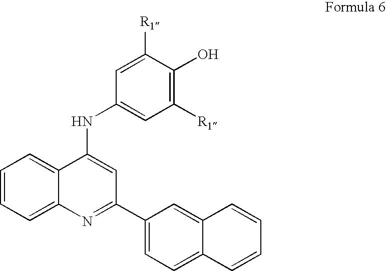
wherein R_{1″} is selected from morpholinomethyl, piperidinomethyl, pyrrolidinomethyl, and N-methylpiperazinomethyl.

19. The method of claim 18, wherein R_{1″} is N-methylpiperazinomethyl.

20. The method of any one of claims 1-6, wherein the small molecule antagonist of immunostimulatory CpG nucleic acids is chosen from bafilomycin A, monensin, concanamycin B, and ammonium chloride.

21. The method of any one of claims 1-6, wherein the nucleic acid-containing immune complex comprises a CpG nucleic acid.

22. The method of any one of claims 1-6, wherein the nucleic acid-containing immune complex comprises a bacterial nucleic acid.

23. The method of any one of claims 1-6, wherein the nucleic acid-containing immune complex comprises a host nucleic acid.

24. The method of any one of claims 1-6, wherein the nucleic acid-containing immune complex comprises DNA.


SEQUENCE LISTING (30 sequences)

SEQ ID NO: 1 - DNA, 18 bp, Artificial sequence
  Synthetic oligonucleotide
gtgccggggtctccgggc

SEQ ID NO: 2 - DNA, 18 bp, Artificial sequence
  Synthetic oligonucleotide
gctgtggggcggctcctg

SEQ ID NO: 3 - DNA, 20 bp, Artificial sequence
  Synthetic oligonucleotide
ggggtcaacgttgagggggg

SEQ ID NO: 4 - DNA, 9 bp, Artificial sequence
  Synthetic oligonucleotide
ggggagggt

SEQ ID NO: 5 - DNA, 9 bp, Artificial sequence
  Synthetic oligonucleotide
ggggagggg

SEQ ID NO: 6 - DNA, 15 bp, Artificial sequence
  Synthetic oligonucleotide
cacgttgaggggcat

SEQ ID NO: 7 - DNA, 15 bp, Artificial sequence
  Synthetic oligonucleotide
tcctggcggggaagt

SEQ ID NO: 8 - DNA, 15 bp, Artificial sequence
  Synthetic oligonucleotide
tcctggaggggaagt

SEQ ID NO: 9 - DNA, 27 bp, Artificial sequence
  Synthetic oligonucleotide
ggctccggggagggaatttttgtctat

SEQ ID NO: 10 - DNA, 15 bp, Artificial sequence
  Synthetic oligonucleotide
tcctgccggggaagt

SEQ ID NO: 11 - DNA, 15 bp, Artificial sequence
  Synthetic oligonucleotide
tcctgcaggggaagt

SEQ ID NO: 12 - DNA, 15 bp, Artificial sequence
  Synthetic oligonucleotide
tcctgaaggggaagt

SEQ ID NO: 13 - DNA, 15 bp, Artificial sequence
  Synthetic oligonucleotide
tcctggcgggcaagt

SEQ ID NO: 14 - DNA, 15 bp, Artificial sequence
  Synthetic oligonucleotide
tcctggcgggtaagt

SEQ ID NO: 15 - DNA, 15 bp, Artificial sequence
  Synthetic oligonucleotide
tcctggcgggaaagt

SEQ ID NO: 16 - DNA, 15 bp, Artificial sequence
  Synthetic oligonucleotide
tccgggcggggaagt

SEQ ID NO: 17 - DNA, 15 bp, Artificial sequence
  Synthetic oligonucleotide
tcggggcggggaagt

SEQ ID NO: 18 - DNA, 15 bp, Artificial sequence
  Synthetic oligonucleotide
tcccggcggggaagt

SEQ ID NO: 19 - DNA, 15 bp, Artificial sequence
  Synthetic oligonucleotide
gggggacgttggggg

SEQ ID NO: 20 - DNA, 66 bp, Artificial sequence
  Synthetic oligonucleotide
nnnnnnnnnnnnnnnnnnnngggnnnnnnnnnnnnnnnnnnnngggnnnnnnnnnnnnnnnnnnnn

SEQ ID NO: 21 - DNA, 3352 bp, Homo sapiens
aggctggtataaaaatcttacttcctctattctctgagccgctgctgcccctgtgggaagggacctcgagtgtgaagcatccttccctgtagctgctgtccagtctgcccgccagaccctctggagaagcccctgccccccagcatgggtttctgccgcagcgccctgcacccgctgtctctcctggtgcaggccatcatgctggccatgaccctggccctgggtaccttgcctgccttcctaccctgtgagctccagccccacggcctggtgaactgcaactggctgttcctgaagtctgtgccccacttctccatggcagcaccccgtggcaatgtcaccagcctttccttgtcctccaaccgcatccaccacctccatgattctgactttgcccacctgcccagcctgcggcatctcaacctcaagtggaactgcccgccggttggcctcagccccatgcacttcccctgccacatgaccatcgagcccagcaccttcttggctgtgcccaccctggaagagctaaacctgagctacaacaacatcatgactgtgcctgcgctgcccaaatccctcatatccctgtccctcagccataccaacatcctgatgctagactctgccagcctcgccggcctgcatgccctgcgcttcctattcatggacggcaactgttattacaagaacccctgcaggcaggcactggaggtggccccgggtgccctccttggcctgggcaacctcacccacctgtcactcaagtacaacaacctcactgtggtgccccgcaacctgccttccagcctggagtatctgctgttgtcctacaaccgcatcgtcaaactggcgcctgaggacctggccaatctgaccgccctgcgtgtgctcgatgtgggcggaaattgccgccgctgcgaccacgctcccaacccctgcatggagtgccctcgtcacttcccccagctacatcccgataccttcagccacctgagccgtcttgaaggcctggtgttgaaggacagttctctctcctggctgaatgccagttggttccgtgggctgggaaacctccgagtgctggacctgagtgagaacttcctctacaaatgcatcactaaaaccaaggccttccagggcctaacacagctgcgcaagcttaacctgtccttcaattaccaaaagagggtgtcctttgcccacctgtctctggccccttccttcgggagcctggtcgccctgaaggagctggacatgcacggcatcttcttccgctcactcgatgagaccacgctccggccactggcccgcctgcccatgctccagactctgcgtctgcagatgaacttcatcaaccaggcccagctcggcatcttcagggccttccctggcctgcgctacgtggacctgtcggacaaccgcatcagcggagcttcggagctgacagccaccatgggggaggcagatggaggggagaaggtctggctgcagcctggggaccttgctccggccccagtggacactcccagctctgaagacttcaggcccaactgcagcaccctcaacttcaccttggatctgtcacggaacaacctggtgaccgtgcagccggagatgtttgcccagctctcgcacctgcagtgcctgcgcctgagccacaactgcatctcgcaggcagtcaatggctcccagttcctgccgctgaccggtctgcaggtgctagacctgtcccgcaataagctggacctctaccacgagcactcattcacggagctaccgcgactggaggccctggacctcagctacaacagccagccctttggcatgcagggcgtgggccacaacttcagcttcgtggctcacctgcgcaccctgcgccacctcagcctggcccacaacaacatccacagccaagtgtcccagcagctctgcagtacgtcgctgcgggccctggacttcagcggcaatgcactgggccatatgtgggccgagggagacctctatctgcacttcttccaaggcctgagcggtttgatctggctggacttgtcccagaaccgcctgcacaccctcctgccccaaaccctgcgcaacctccccaagagcctacaggtgctgcgtctccgtgacaattacctggccttctttaagtggtggagcctccacttcctgcccaaactggaagtcctcgacctggcaggaaaccggctgaaggccctgaccaatggcagcctgcctgctggcacccggctccggaggctggatgtcagctgcaacagcatcagcttcgtggcccccggcttcttttccaaggccaaggagctgcgagagctcaaccttagcgccaacgccctcaagacagtggaccactcctggtttgggcccctggcgagtgccctgcaaatactagatgtaagcgccaaccctctgcactgcgcctgtggggcggcctttatggacttcctgctggaggtgcaggctgccgtgcccggtctgcccagccgggtgaagtgtggcagtccgggccagctccagggcctcagcatctttgcacaggacctgcgcctctgcctggatgaggccctctcctgggactgtttcgccctctcgctgctggctgtggctctgggcctgggtgtgcccatgctgcatcacctctgtggctgggacctctggtactgcttccacctgtgcctggcctggcttccctggcgggggcggcaaagtgggcgagatgaggatgccctgccctacgatgccttcgtggtcttcgacaaaacgcagagcgcagtggcagactgggtgtacaacgagcttcgggggcagctggaggagtgccgtgggcgctgggcactccgcctgtgcctggaggaacgcgactggctgcctggcaaaaccctctttgagaacctgtgggcctcggtctatggcagccgcaagacgctgtttgtgctggcccacacggaccgggtcagtggtctcttgcgcgccagcttcctgctggcccagcagcgcctgctggaggaccgcaaggacgtcgtggtgctggtgatcctgagccctgacggccgccgctcccgctacgtgcggctgcgccagcgcctctgccgccagagtgtcctcctctggccccaccagcccagtggtcagcgcagcttctgggcccagctgggcatggccctgaccagggacaaccaccacttctataaccggaacttctgccagggacccacggccgaatagccgtgagccggaatcctgcacggtgccacctccacactcacctcacctctgcctgcctggtctgaccctcccctgctcgcctccctcaccccacacctgacacagagca

SEQ ID NO: 22 - protein, 1032 aa, Homo sapiens [sequence omitted]

SEQ ID NO: 23 - DNA, 3200 bp, Mus musculus
tgtcagagggagcctcgggagaatcctccatctcccaacatggttctccgtcgaaggactctgcaccccttgtccctcctggtacaggctgcagtgctggctgagactctggccctgggtaccctgcctgccttcctaccctgtgagctgaagcctcatggcctggtggactgcaattggctgttcctgaagtctgtaccccgtttctctgcggcagcatcctgctccaacatcacccgcctctccttgatctccaaccgtatccaccacctgcacaactccgacttcgtccacctgtccaacctgcggcagctgaacctcaagtggaactgtccacccactggccttagccccctgcacttctcttgccacatgaccattgagcccagaaccttcctggctatgcgtacactggaggagctgaacctgagctataatggtatcaccactgtgccccgactgcccagctccctggtgaatctgagcctgagccacaccaacatcctggttctagatgctaacagcctcgccggcctatacagcctgcgcgttctcttcatggacgggaactgctactacaagaacccctgcacaggagcggtgaaggtgaccccaggcgccctcctgggcctgagcaatctcacccatctgtctctgaagtataacaacctcacaaaggtgccccgccaactgccccccagcctggagtacctcctggtgtcctataacctcattgtcaagctggggcctgaagacctggccaatctgacctcccttcgagtacttgatgtgggtgggaattgccgtcgctgcgaccatgcccccaatccctgtatagaatgtggccaaaagtccctccacctgcaccctgagaccttccatcacctgagccatctggaaggcctggtgctgaaggacagctctctccatacactgaactcttcctggttccaaggtctggtcaacctctcggtgctggacctaagcgagaactttctctatgaaagcatcaaccacaccaatgcctttcagaacctaacccgcctgcgcaagctcaacctgtccttcaattaccgcaagaaggtatcctttgcccgcctccacctggcaagttccttcaagaacctggtgtcactgcaggagctgaacatgaacggcatcttcttccgctcgctcaacaagtacacgctcagatggctggccgatctgcccaaactccacactctgcatcttcaaatgaacttcatcaaccaggcacagctcagcatctttggtaccttccgagcccttcgctttgtggacttgtcagacaatcgcatcagtgggccttcaacgctgtcagaagccacccctgaagaggcagatgatgcagagcaggaggagctgttgtctgcggatcctcacccagctccactgagcacccctgcttctaagaacttcatggacaggtgtaagaacttcaagttcaccatggacctgtctcggaacaacctggtgactatcaagccagagatgtttgtcaatctctcacgcctccagtgtcttagcctgagccacaactccattgcacaggctgtcaatggctctcagttcctgccgctgactaatctgcaggtgctggacctgtcccataacaaactggacttgtaccactggaaatcgttcagtgagctaccacagttgcaggccctggacctgagctacaacagccagccctttagcatgaagggtataggccacaatttcagttttgtggcccatctgtccatgctacacagccttagcctggcacacaatgacattcatacccgtgtgtcctcacatctcaacagcaactcagtgaggtttcttgacttcagcggcaacggtatgggccgcatgtgggatgaggggggcctttatctccatttcttccaaggcctgagtggcctgctgaagctggacctgtctcaaaataacctgcatatcctccggccccagaaccttgacaacctccccaagagcctgaagctgctgagcctccgagacaactacctatctttctttaactggaccagtctgtccttcctgcccaacctggaagtcctagacctggcaggcaaccagctaaaggccctgaccaatggcaccctgcctaatggcaccctcctccagaaactggatgtcagcagcaacagtatcgtctctgtggtcccagccttcttcgctctggcggtcgagctgaaagaggtcaacctcagccacaacattctcaagacggtggatcgctcctggtttgggcccattgtgatgaacctgacagttctagacgtgagaagcaaccctctgcactgtgcctgtggggcagccttcgtagacttactgttggaggtgcagaccaaggtgcctggcctggctaatggtgtgaagtgtggcagccccggccagctgcagggccgtagcatcttcgcacaggacctgcggctgtgcctggatgaggtcctctcttgggactgctttggcctttcactcttggctgtggccgtgggcatggtggtgcctatactgcaccatctctgcggctgggacgtctggtactgttttcatctgtgcctggcatggctacctttgctggcccgcagccgacgcagcgcccaagctctcccctatgatgccttcgtggtgttcgataaggcacagagcgcagttgcggactgggtgtataacgagctgcgggtgcggctggaggagcggcgcggtcgccgagccctacgcttgtgtctggaggaccgagattggctgcctggccagacgctcttcgagaacctctgggcttccatctatgggagccgcaagactctatttgtgctggcccacacggaccgcgtcagtggcctcctgcgcaccagcttcctgctggctcagcagcgcctgttggaagaccgcaaggacgtggtggtgttggtgatcctgcgtccggatgcccaccgctcccgctatgtgcgactgcgccagcgtctctgccgccagagtgtgctcttctggccccagcagcccaacgggcaggggggcttctgggcccagctgagtacagccctgactagggacaaccgccacttctataaccagaacttctgccggggacctacagcagaatagctcagagcaacagctggaaacagctgcatcttcatgcctggttcccgagttgctctgcctgc

SEQ ID NO: 24 - protein, 1032 aa, Mus musculus
MVLRRRTLHPLSLLVQAAVLAETLALGTLPAFLPCELKPHGLVDCNWLFLKSVPRFSAAASCSNITRLSLISNRIHHLHNSDFVHLSNLRQLNLKWNCPPTGLSPLHFSCHMTIEPRTFLAMRTLEELNLSYNGITTVPRLPSSLVNLSLSHTNILVLDANSLAGLYSLRVLFMDGNCYYKNPCTGAVKVTPGALLGLSNLTHLSLKYNNLTKVPRQLPPSLEYLLVSYNLIVKLGPEDLANLTSLRVLDVGGNCRRCDHAPNPCIECGQKSLHLHPETFHHLSHLEGLVLKDSSLHTLNSSWFQGLVNLSVLDLSENFLYESINHTNAFQNLTRLRKLNLSFNYRKKVSFARLHLASSFKNLVSLQELNMNGIFFRSLNKYTLRWLADLPKLHTLHLQMNFINQAQLSIFGTFRALRFVDLSDNRISGPSTLSEATPEEADDAEQEELLSADPHPAPLSTPASKNFMDRCKNFKFTMDLSRNNLVTIKPEMFVNLSRLQCLSLSHNSIAQAVNGSQFLPLTNLQVLDLSHNKLDLYHWKSFSELPQLQALDLSYNSQPFSMKGIGHNFSFVAHLSMLHSLSLAHNDIHTRVSSHLNSNSVRFLDFSGNGMGRMWDEGGLYLHFFQGLSGLLKLDLSQNNLHILRPQNLDNLPKSLKLLSLRDNYLSFFNWTSLSFLPNLEVLDLAGNQLKALTNGTLPNGTLLQKLDVSSNSIVSVVPAFFALAVELKEVNLSHNILKTVDRSWFGPIVMNLTVLDVRSNPLHCACGAAFVDLLLEVQTKVPGLANGVKCGSPGQLQGRSIFAQDLRLCLDEVLSWDCFGLSLLAVAVGMVVPILHHLCGWDVWYCFHLCLAWLPLLARSRRSAQALPYDAFVVFDKAQSAVADWVYNELRVRLEERRGRRALRLCLEDRDWLPGQTLFENLWASIYGSRKTLFVLAHTDRVSGLLRTSFLLAQQRLLEDRKDVVVLVILRPDAHRSRYVRLRQRLCRQSVLFWPQQPNGQGGFWAQLSTALTRDNRHFYNQNFCRGPTAE

SEQ ID NO: 25 - DNA, 24 bp, Artificial sequence
  Synthetic oligonucleotide
tcgtcgttttgtcgttttgtcgtt

SEQ ID NO: 26 - DNA, 24 bp, Artificial sequence
  Synthetic oligonucleotide
tgctgcttttgtgcttttgtgctt

SEQ ID NO: 27 - DNA, 24 bp, Artificial sequence
  Synthetic oligonucleotide
tcgtcgttttgtcgttttgtcgtt

SEQ ID NO: 28 - DNA, 20 bp, Artificial sequence
  Synthetic oligonucleotide
tccatgacgttcctgatgct

SEQ ID NO: 29 - DNA, 20 bp, Artificial sequence
  Synthetic oligonucleotide
tccatgagcttcctgatgct

SEQ ID NO: 30 - DNA, 20 bp, Artificial sequence
  Synthetic oligonucleotide
tccatgacgttcctgatgct